(19) United States
(12) Patent Application Publication
(10) Pub. No.: US 2017/0157233 A1
(43) Pub. Date: Jun. 8, 2017

(54) Immunogenic Compositions

(71) Applicant: Sanofi Pasteur Limited, Toronto (CA)

(72) Inventors: Scott Gallichan, Toronto (CA); Belma Ljutic, Toronto (CA); Martina Ochs-Onolemhemhen, Toronto (CA); Marie-Danielle Salha, Toronto (CA); Robert Hopfer, Toronto (CA); Lee-Jah Chang, Toronto (CA)

(73) Assignee: Sanofi Pasteur Limited, Toronto, ON (CA)

(21) Appl. No.: 15/124,417

(22) PCT Filed: Mar. 10, 2015
(86) PCT No.: PCT/IB2015/000985

§ 371 Date: Sep. 8, 2016

Related U.S. Application Data
(60) Provisional application No. 61/950,414, filed on Mar. 10, 2014.

(51) Int. Cl. A61K 39/09

(52) U.S. Cl. CPC A61K 39/092; A61K 2039/55505

(57) ABSTRACT

This disclosure relates to immunogenic compositions comprising an isolated immunogenic S. pneumoniae PcpA polypeptide, at least one additional antigen (such as for example, an isolated immunogenic S. pneumoniae polypeptide selected from the group consisting of the polyhistidine triad family of proteins (e.g., PhtD), and at least one isolated detoxified pneumolysin (e.g., PlyD1) and methods of using these compositions for preventing and treating diseases caused by S. pneumoniae.

### RELATED APPLICATION

This application claims priority to U.S. Ser. No. 61/950,414 filed Mar. 10, 2014, which is incorporated in its entirety into this application.

### FIELD OF DISCLOSURE

The present disclosure relates to the field of immunology and, in particular, to Streptococcus pneumoniae antigens and their use in immunization.

### BACKGROUND INFORMATION

Streptococcus pneumoniae is a rather ubiquitous human pathogen, frequently found in the upper respiratory tract of healthy children and adults. These bacteria can infect several organs including the lungs, the central nervous system (CNS), the middle ear, and the nasal tract and cause a range of diseases (i.e., symptomatic infections) such as for example, sinus infection, otitis media, bronchitis, pneumonia, meningitis, and bacteremia (septicemia). Pneumococcal meningitis, the most severe form of these pneumococcal diseases, is associated with significant mortality and morbidity despite antibiotic treatment (Quagliarello et. al. (1992) N. Engl. J. Med. 327:864-872). Children under the age of two and the elderly are particularly susceptible to symptomatic pneumococcal infections.

Currently, there are two available types of pneumococcal vaccines. The first includes capsular polysaccharides from 23 types of S. pneumoniae, which together represent the capsular types of about 90% of strains causing pneumococcal infection. This vaccine, however, is not very immunogenic in young children, an age group with heightened susceptibility to pneumococcal infection as they do not generate a good immune response to polysaccharide antigens prior to 2 years of age. In adults the vaccine has been shown to be about 60% efficacious against bacteremic pneumonia, but it is less efficacious in adults at higher risk of pneumococcal infection because of age or underlying medical conditions (Fedson, and Musher 2004, “Pneumococcal Polysaccharide Vaccine”, pp. 529-588; In Vaccines. S. A. Plotikin and W. A. Orenstein (eds.), W.B. Saunders and Co., Philadelphia, Pa.; Shapiro et. al., N. Engl. J. Med. 325:1453-1460 (1991)).

The second available type are conjugate vaccines. These vaccines which include serotype specific capsular polysaccharide antigens conjugated to a protein carrier, elicit serotype-specific protection (9). Currently available are 7-valent and 13-valent conjugate vaccines: the 7-valent includes 7 polysaccharide antigens (derived from the capsules of serotypes 4, 6B, 9V, 14, 18C, 19F and 23F) and the 13-valent includes 13 polysaccharide antigens (derived from the capsules of serotypes 1, 3, 5, 6A, 7F and 19A, in addition to those covered by the 7-valent). A 9-valent and 11-valent conjugate vaccine have also been developed and each includes polysaccharides specific for serotypes not covered by the 7-valent (i.e., serotypes 1 and 5 in the 9-valent and types 3 and 7F in the 11-valent).

The manufacture of conjugate vaccines is complex and costly due in part to the need to produce 7 (or 9 or 11) different polysaccharides each conjugated to the protein carrier. Such vaccines also do not do a good job of covering infections in the developing world where serotypes of Streptococcus pneumoniae not covered by the conjugate vaccines are very common (Di Fabio et al., Pediatr. Infect. Dis. J. 20:959-967 (2001); Mulholland, Trop. Med. Int. Health 10:497-500 (2005)). The use of the 7-valent conjugate vaccine has also been shown to have led to an increase in colonization and disease with strains of capsule types not represented by the 7 polysaccharides included in the vaccine (Bogaert et al., Lancet Infect. Dis. 4:144-154 (2004); Eskola et al., N. Engl. J. Med. 344-403-409 (2001); Mbelle et al., J. Infect. Dis. 180:1171-1176 (1999)).

As an alternative to the polysaccharide based vaccines currently available, a number of S. pneumoniae antigens have been suggested as possible candidates for a protein-based vaccine against S. pneumoniae. To date, however, no such vaccine is currently available on the market, especially for infant human beings. Therefore, a need remains for effective treatments for S. pneumoniae.

### SUMMARY OF THE DISCLOSURE

Compositions and methods for immunizing human beings, including infant human beings, are described in this disclosure. This disclosure provides compositions comprising an immunogen corresponding to each of PhtD (e.g., at least about 80% sequence identity to the amino acid sequence as set forth in SEQ ID NO:1), PcpA (e.g., having at least about 80% sequence identity to the amino acid sequence as set forth in SEQ ID NO:2), and PlyD1 (e.g., having at least about 80% sequence identity to the amino acid sequence as set forth in SEQ ID NO:9 or 10, and/or being a wild-type pneumolysis protein comprising amino acid substitutions at positions 65, 293 and 428 of the wild type sequence. In some embodiments, the compositions are immunogenic and induces and/or enhance the production of antibodies having specificity for each of PcpA, PhtD, and PlyD1 upon administration to a host. In some embodiments, the composition may comprise about 10 to about 50 μg of each immunogen, especially about 10 μg, about 25 μg, or about 50 μg. In some embodiments, the composition further comprises at least one adjuvant such as an aluminum compound. In some embodiments, administration of the compositions to a host induces an immune response against and/or protects a host against infection by S. pneumonia. In some embodiments, the human being may be between about 18 to about 50 years old or older, about 12 to about 13 months old, or about six to about 14 weeks old or older. In certain embodiments, the administration of the composition may be performed when the human being is about six weeks old (e.g., an initial administration of a multi-dose regimen comprising, for instance, two, three or more administrations). In some embodiments, each dose comprises a 1:1:1 (w/w) ratio of each immuogen (e.g., about 10 μg, 25 μg or 50 μg). In some embodiments, each dose comprises 1:1:2 (w/w) ratio of PcpA, PhtD and detoxified pneumolysin (e.g., 25 μg each of PcpA and PhtD, and about 50 μg of the detoxified pneumolysin). Thus, in some embodiments, methods for inducing antibodies against PcpA and PhtD, and neutralizing antibodies against a detoxified pneumolysin, in a human infant, by administering to the infant a composition comprising at least one adjuvant and immunogens corresponding to PcpA, PhtD, and a detoxified pneumolysin, the immunogens being in the composition at a ratio of about 1:1:1 (w/w) or 1:1:2 (w/w), are provided. In some embodiments, the methods may induce a neutralizing and/or toxin-neutralizing immune response against S. pneumoniae in a human being. In some such embodiments, the methods may comprise administering one or more doses of about 25 μg/dose or more of detoxified pneumolysin thereto. In some embodiments, the neutralizing antibodies against pnuemolysin are detected following the first, second, or third administration. The features and advantages of the disclosure will be apparent from the following Detailed Description, the Drawings and the Claims. Other embodiments are also contemplated, as would be apparent to those of ordinary skill in the art.

### BRIEF DESCRIPTION OF FIGURES

The present disclosure will be further understood from the following description with reference to the drawings, in which:

FIG. 1. Geometric mean concentrations of antibodies in serum of human beings vaccinated with trivalent composition of PcpA, PhtD and PlyD1.

FIG. 2. Serum neutralization assay.

FIG. 3. Results of serum neutralization assay using serum of vaccinated infants.

FIG. 4. Passive immunization assay.

FIG. 5. Results of passive immunization assay using serum of vaccinated infants.

FIG. 6. Predicted probability of survival for high dose placebo and high dose (50 μg per antigen) vaccinated subjects.

FIG. 7. Percentage of subjects showing greater than or equal to two-fold increase in antibody levels following vaccination.

### DETAILED DESCRIPTION OF DISCLOSURE

Immunogenic compositions and methods for eliciting an immune response against Streptococcus infections (such as e.g., S. pneumoniae) are described. Pharmaceutical compositions (e.g., vaccine compositions), including one or more immunogenic PcpA polypeptides, PhtX polypeptides and/or detoxified pneumolysin proteins are provided. Optionally, the compositions can include an adjuvant. The compositions may also include one or more pharmaceutically acceptable excipients, which increase the thermal stability of the polypeptides/proteins relative to a composition lacking the one or more pharmaceutically acceptable excipients. In one example, the one or more pharmaceutically acceptable excipients increase the thermal stability of PcpA, PhtX and/or detoxified pneumolysin protein by 0.5° C. or more, relative to a composition lacking the one or more pharmaceutically acceptable excipients. The compositions can be in liquid form, dry powder form, freeze dried, spray dried and or foam dried. The one or more pharmaceutically acceptable excipients can be for example, selected from the group consisting of buffers, tonicity agents, simple carbohydrates, sugars, carbohydrate polymers, amino acids, oligopeptides, polyamino acids, polyhydric alcohols and ethers thereof, detergents, lipids, surfactants, antioxidants, salts, human serum albumin, gelatins, formaldehyde, or combinations thereof.

Also provided are methods of inducing an immune response to S. pneumoniae in a subject, which involve administering to the subject a composition as described herein. Use of the compositions of the disclosure in inducing an immune response to S. pneumoniae in a subject, or in preparation of medicaments for use in this purpose is also provided.

The disclosure provides several advantages. For example, administration of the compositions of the present disclosure to a subject elicits an immune response against infections by a number of strains of S. pneumoniae. In addition, the multivalent compositions of the present disclosure include specific combinations of immunogenic polypeptides of S. pneumoniae which when administered do not experience antigenic interference and may provide additive effects. Use of the excipients described herein can result in increased thermal stability of the polypeptides/proteins within the compositions.

Compositions and methods for eliciting an immune response against S. pneumoniae and for treating and preventing disease caused by S. pneumoniae in human beings are provided. Provided are immunogenic compositions comprising immunogenic PcpA polypeptides and/or immunogenic polypeptides of the polyhistidine triad family (PhtX: PhtA, PhtB, PhtD, PhtE), and detoxified pneumolysin as well as methods for their production and their use. Methods include passive and active immunization approaches, which include administration (e.g., subcutaneous, intramuscular) of immunogenic compositions comprising one or more substantially purified Streptococcal (e.g., S. pneumoniae) polypeptides, antibodies to the polypeptides themselves, or a combination thereof. The disclosure also includes Streptococcus sp. (e.g., S. pneumoniae) polypeptides, immunogenic compositions (e.g., vaccines) comprising Streptococcal polypeptides, methods of producing such compositions, and methods of producing Streptococcal (e.g., S. pneumoniae) antibodies. These methods and compositions are described further, below.

The compositions of the disclosure include one, two, three or more immunogenic polypeptides. The compositions may include for example, in combination, an immunogenic polypeptide of PcpA; an immunogenic polypeptide of a member of the polyhistidine triad family of proteins (e.g., PhtA, PhtB, PhtD, and PhtE, referenced herein as PhtX proteins); and a detoxified pneumolysin polypeptide Immunogenic fragments and fusions of these polypeptides may also be included in the compositions (e.g., a fusion of PhtB and PhtE). These immunogenic polypeptides may optionally be used in combination with pneumococcal saccharides or other pneumococcal polypeptides.

In one multi-component example, the immunogenic composition includes an immunogenic PcpA polypeptide and one or more immunogenic PhtX polypeptides. A preferred embodiment of such a composition comprises an immunogenic PhtD polypeptide, an immunogenic PcpA polypeptide and detoxified pneumolysin. Certain embodiments of the immunogenic composition (in e.g., bivalent and trivalent form) are described in the Examples herein.

### Polypeptides

Immunogenic PcpA polypeptides comprise the full-length PcpA amino acid sequence (in the presence or absence of the signal sequence), fragments thereof, and variants thereof. PcpA polypeptides suitable for use in the compositions described herein include, for example, those of GenBank Accession No. CAB04758 from S. pneumoniae strain B6, GenBank Accession No. AAK76194 from S. pneumoniae strain TIGR4 and GenBank Accession No. NP_359536 from S. pneumoniae strain R6, and those from S. pneumoniae strain 14453.

The amino acid sequence of full length PcpA in the S. pneumoniae 14453 genome is SEQ ID NO. 2. Preferred PcpA polypeptides for use with the disclosure comprise an amino acid sequence having 50% or more identity (e.g, 60, 65, 70, 75, 80, 85, 85, 90, 91, 92, 93, 94, 95, 96, 97, 98, 99, 99.5% or more) to SEQ ID NO:2 or SEQ ID NO:7. Preferred polypeptides for use with the disclosure comprise a fragment of at least 8, 9, 10, 12, 14, 16, 18, 20, 25, 30, 35, 40, 50, 60, 70, 80, 90, 100, 150, 200, 250 or more consecutive amino acids of SEQ ID NO:2. Preferred fragments comprise an epitope from SEQ ID NO. 2. Other preferred fragments lack one or more amino acids from the N-terminus of SEQ ID NO. 2 (e.g., 1, 2, 3, 4, 5, 6, 7, 8, 9, 10, 15, 20, 25 or more) and/or one or more amino acids from the C-terminus of SEQ ID NO:2 while retaining at least one epitope of SEQ ID NO:2. Further preferred fragments lack the signal sequence from the N-terminus of SEQ ID NO:2. A preferred PcpA polypeptide is SEQ ID NO:7.

Optionally, immunogenic polypeptides of PcpA comprise one or more leucine rich regions (LRRs). These LLRs are present in naturally occurring PcpA or have about 60 to about 99% sequence identity, including, for example, 80%, 85%, 90% or 95% sequence identity to the naturally occurring LRRs. LRRs in the mature PcpA protein (i.e., the protein lacking the signal peptide) can be found in certain sequences disclosed in WO 2008/022302 (e.g., SEQ ID NOs:1, 2, 41 and 45 of WO 2008/022302).

An immunogenic polypeptide of PcpA optionally lacks the choline binding domain anchor sequence typically present in the naturally occurring mature PcpA protein. The naturally occurring sequence of the choline binding anchor of the mature PcpA protein is disclosed in WO 2008/022302 as SEQ ID NO:52. More particularly, an immunogenic polypeptide comprises an N-terminal region of naturally occurring PcpA with one or more amino acid substitutions and about 60 to about 99% sequence identity or any identity in between, e.g. 80, 85, 90 and 95% identity, to the naturally occurring PcpA. The N-terminal region may comprise the amino acid sequence of SEQ ID NO: 2 (or SEQ ID NOs: 1, 2, 3, 4, 41 or 45 of WO2008/022302), in the presence or absence of one or more conservative amino acid substitutions and in the presence or absence of the signal sequence. The N-terminal region may comprise an amino acid sequence having about 60 to about 99% sequence identity (or any identity in between 80 to 99% identity) to SEQ ID NOs: 2 or 7 (set out in the Sequence Listing herein) or SEQ ID NOs:1, 2, 3, 4, or 41 of WO2008/022302.

Immunogenic fragments of SEQ ID NOs: 2 and 7 comprise 5, 10, 20, 30, 40, 50, 60, 70, 80, 90, 100, 110, 120, 130, 140, 150, 160, 170, 180, 190 and 191 amino acid residues of SEQ ID NOs: 2 and 7 or any number of amino acid residues between 5 and 191. Examples of immunogenic fragments of PcpA are disclosed in WO 2008/022302.

Optionally, immunogenic polypeptides of PcpA lack the LRRs. Examples of immunogenic polypeptides lacking the LRR are disclosed in WO 2008/022302 as SEQ ID NO:29, SEQ ID NO:30, and SEQ ID NO:31.

Immunogenic PhtX polypeptides suitable for the compositions of the disclosure comprise the full-length PhtA, PhtB, PhtD or PhtE amino acid sequence (in the presence or absence of the signal sequence), immunogenic fragments thereof, variants thereof and fusion proteins thereof. PhtD polypeptides suitable for use in the compositions described herein include, for example, those of GenBank Accession Nos. AAK06760, YP816370 and NP35851, among others. The amino acid sequence of full length PhtD in the S. pneumoniae 14453 genome is SEQ ID NO:1. A preferred polypeptide of PhtD (derived from the S. pneumoniae 14453 genome) is SEQ ID NO:5.

The immunogenic fragments of PhtX polypeptides of the present disclosure are capable of eliciting an immune response specific for the corresponding full length mature amino acid sequence.

Immunogenic PhtX (e.g., PhtD) polypeptides include the full length protein with the signal sequence attached, the mature full length protein with the signal peptide (e.g., 20 amino acids at N-terminus) removed, variants of PhtX (naturally occurring or otherwise, e.g, synthetically derived) and immunogenic fragments of PhtX (e.g, fragments comprising at least 15 or 20 contiguous amino acids present in the naturally occurring mature PhtX protein).

Examples of immunogenic fragments of PhtD are disclosed in US 2010/0297133A1.

Preferred PhtD polypeptides for use with the disclosure comprise an amino acid sequence having 50% or more identity (e.g., 60, 65, 70, 75, 80, 85, 85, 90, 91, 92, 93, 94, 95, 96, 97, 98, 99, 99.5% or more) to SEQ ID NO:1 or to SEQ ID NO:5. Preferred polypeptides for use with the disclosure comprise a fragment of at least 8, 9, 10, 12, 14, 16, 18, 20, 25, 30, 35, 40, 50, 60, 70, 80, 90, 100, 150, 200, 250 or more consecutive amino acids of SEQ ID NO:1. Preferred fragments comprise an epitope from SEQ ID NO:1 or to SEQ ID NO:5. Other preferred fragments lack one or more amino acids from the N-terminus of SEQ ID NO:1 (e.g., 1, 2, 3, 4, 5, 6, 7, 8, 9, 10, 15, 20, 25 or more) and/or one or amino acids from the C-terminus of SEQ ID NO:1 while retaining at least one epitope of SEQ ID NO:1. Further preferred fragments lack the signal sequence from the N-terminus of SEQ ID NO:1. A preferred PhtD polypeptide is SEQ ID NO:5.

Pneumolysin (Ply) is a cytolytic-activating toxin implicated in multiple steps of pneumococcal pathogenesis, including the inhibition of ciliary beating and the disruption of tight junctions between epithelial cells (Hirst et al. Clinical and Experimental Immunology (2004)). Several pneumolysins are known and (following detoxification) would be suitable for use in the compositions described herein including, for example GenBank Accession Nos. Q04IN8, POC2J9, Q7ZAK5, and AB021381, among others. In one embodiment, Ply has the amino acid sequence shown in SEQ ID NO:10.

Immunogenic pneumolysin polypeptides for use with the disclosure include the full length protein with the signal sequence attached, the mature full length protein with the signal peptide removed, variants of pneumolysin (naturally occurring or otherwise, e.g., synthetically derived) and immunogenic fragments of pneumolysin (e.g, fragments comprising at least 15 or 20 contiguous amino acids present in the naturally occurring mature pneumolysin protein).

Immunogenic variants and fragments of the immunogenic pneumolysin polypeptides of the present disclosure are capable of eliciting an immune response specific for the corresponding full length mature amino acid sequence. The immunogenic pneumolysin polypeptides of the present disclosure are detoxified; that is, they lack or have reduced toxicity as compared to the mature wild-type pneumolysin protein produced and released by S. pneumoniae. The immunogenic pneumolysin polypeptides of the present disclosure may be detoxified for example, chemically (e.g., using formaldehyde treatment) or genetically (e.g., recombinantly produced in a mutated form).

Preferred examples of the immunogenic detoxified pneumolysin for use in the present disclosure are disclosed in US 2011/0287046A1. As disclosed in that application, the detoxified pneumolysin may be a mutant pneumolysin protein comprising amino acid substitutions at positions 65, 293 and 428 of the wild type sequence. In a preferred detoxified pneumolysin protein, the three amino acid substitutions comprise T₆₅→C, G₂₉₃→C, and C₄₂₈→A. A preferred immunogenic and detoxified pneumolysin polypeptide is SEQ ID NO:9.

Preferred pneumoysin polypeptides for use with the disclosure comprise an amino acid sequence having 50% or more identity (e.g., 60, 65, 70, 75, 80, 85, 85, 90, 91, 92, 93, 94, 95, 96, 97, 98, 99, 99.5% or more) to SEQ ID NO:9 or to SEQ ID NO:10. Preferred polypeptides for use with the disclosure comprise a fragment of at least 8, 9, 10, 12, 14, 16, 18, 20, 25, 30, 35, 40, 50, 60, 70, 80, 90, 100, 150, 200, 250 or more consecutive amino acids of SEQ ID NO:9 or 10. Preferred fragments comprise an epitope from SEQ ID NO.9 or to SEQ ID NO:10. Other preferred fragments lack one or more amino acids from the N-terminus of SEQ ID NO. 9 or 10 (e.g., 1, 2, 3, 4, 5, 6, 7, 8, 9, 10, 15, 20, 25 or more) and/or one or amino acids from the C-terminus of SEQ ID NO:9 or 10 while retaining at least one epitope of SEQ ID NO:9 or 10. Further preferred fragments lack the signal sequence from the N-terminus of SEQ ID NO:10.

The immunogenic polypeptides of PcpA, PhtX (e.g., PhtD), and pneumolysin described herein, and fragments thereof, include variants. Such variants of the immunogenic polypeptides described herein are selected for their immunogenic capacity using methods well known in the art and may comprise one or more conservative amino acid modifications. Variants of the immunogenic polypeptides (of PcpA, PhtD, pneumolysin) include amino acid sequence having about 60 to about 99% sequence identity (or any identity in between 60 and 99% identity) to the disclosed sequences (i.e., SEQ ID NO:2 or 7 (PcpA); SEQ ID NO:1 or 5 (PhtD); SEQ ID NO: 9 or 10 (Ply)). Amino acid sequence modifications include substitutional, insertional or deletional changes. Substitutions, deletions, insertions or any combination thereof may be combined in a single variant so long as the variant is an immunogenic polypeptide. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in a recombinant cell culture. Techniques for making substitution mutations are predetermined sites in DNA having a known sequence are well known and include, but are not limited to, M13 primer mutagenesis and PCR mutagenesis. Amino acid substitutions are typically of single residues but can occur at a number of different locations at once. Substitutional variants are those in which at least one residue has been removed and a different residue inserted in its place. Such substitutions generally are made in accordance with the following Table and are referred to as conservative substitutions. Others are well known to those of skill in the art.

As used herein, the amino acid substitution may be conservative or non-conservative. Conservative amino acid substitutions may involve a substitution of a native amino acid residue with a non-native residue such that there is little or no effect on the size, polarity, charge, hydrophobicity, or hydrophilicity of the amino acid residue at that position and, in particular, does not result in decreased immunogenicity. Suitable conservative amino acid substitutions are shown in the Table 1 below.

TABLE 1

| | | Preferred |
|---|---|---|
| Original | | Conservative |
| Residues | Exemplary Conservative Substitutions | Substitution |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

The specific amino acid substitution selected may depend on the location of the site selected. In certain embodiments, nucleotides encoding polypeptides and/or fragments are substituted based on the degeneracy of the genetic code (i.e, consistent with the “Wobble” hypothesis). Where the nucleic acid is a recombinant DNA molecule useful for expressing a polypeptide in a cell (e.g., an expression vector), a Wobble-type substitution will result in the expression of a polypeptide with the same amino acid sequence as that originally encoded by the DNA molecule. As described above, however, substitutions may be conservative, or non-conservative, or any combination thereof. A skilled artisan will be able to determine suitable variants of the polypeptides and/or fragments provided herein using well-known techniques.

Analogs can differ from naturally occurring S. pneumoniae polypeptides in amino acid sequence and/or by virtue of non-sequence modifications. Non-sequence modifications include changes in acetylation, methylation, phosphyorylation, carboxylation, or glycosylation. A “modification” of a polypeptide of the present disclosure includes polypeptides (or analogs thereof, such as, e.g. fragments thereof) that are chemically or enzymatically derived at one or more constituent amino acid. Such modifications can include, for example, side chain modifications, backbone modifications, and N- and C-terminal modifications such as, for example, acetylation, hydroxylation, methylation, amidation, and the attachment of carbonhydrate or lipid moieties, cofactors, and the like, and combinations thereof. Modified polypeptides of the disclosure may retain the biological activity of the unmodified polypeptides or may exhibit a reduced or increased biological activity.

Structural similarity of two polypeptides can be determined by aligning the residues of the two polypeptides (for example, a candidate polypeptide and the polypeptide of, for example, SEQ ID NO: 2) to optimize the number of identical amino acids along the length of their sequences; gaps in either or both sequences are permitted in making the alignment in order to optimize the number of identical amino acids, although the amino acids in each sequence must nonetheless remain in their proper order. A candidate polypeptide is the polypeptide being compared to the reference polypeptide. A candidate polypeptide can be isolated, for example, from a microbe, or can be produced using recombinant techniques, or chemically or enzymatically synthesized.

A pair-wise comparison analysis of amino acids sequences can be carried out using a global algorithm, for example, Needleman-Wunsch. Alternatively, polypeptides may be compared using a local alignment algorithm such as the Blastp program of the BLAST 2 search algorithm, as described by Tatiana et al., (FEMS Microbiol. Lett, 174 247-250 (1999), and available on the National Centre for Biotechnology Information (NCBI) website. The default values for all BLAST 2 search parameters may be used, including matrix=BLOSUM62; open gap penalty=11, extension gap penalty=1, gap x dropoff=50, expect 10, wordsize=3, and filter on. The Smith and Waterman algorithm is another local alignment tool that can be used (1988).

In the comparison of two amino acid sequences, structural similarly may be referred to by percent “identity” or may be referred to by percent “similarity.” “Identity” refers to the presence of identical amino acids. “Similarity” refers to the presences of not only identical amino acid but also the presence of conservative substitutions. A conservative substitution for an amino acid in a polypeptide of the disclosure may be selected from other members of the class to which the amino acid belongs, shown on Table 1.

The nucleic acids encoding the immunogenic polypeptides may be isolated for example, but without limitation from wild type or mutant S. pneumoniae cells or alternatively, may be obtained directly from the DNA of an S. pneumoniae strain carrying the applicable DNA gene (e.g., pcpA, phtD, ply), by using the polymerase chain reaction (PCR) or by using alternative standard techniques that are recognized by one skilled in the art. Possible strains of use include for example, S. pneumoniae strains TIGR4 and 14453. In preferred embodiments the polypeptides are recombinantly derived from S. pneumoniae strain 14453. Preferred examples of the isolated nucleic acid molecules of the present disclosure have nucleic acid sequences set out in SEQ ID NOs: 3, 4, 6 and 8. Sequence-conservative variants and function-conservative variants of these sequences are encompassed by the present disclosure.

The polypeptides of the present disclosure can be produced using standard molecular biology techniques and expression systems (see for example, Molecular Cloning: A Laboratory Manual, Third Edition by Sambrook et. al., Cold Spring Harbor Press, 2001). For example, a fragment of a gene that encodes an immunogenic polypeptide may be isolated and the polynucleotide encoding the immunogenic polypeptide may be cloned into any commercially available expression vector (such as, e.g., pBR322, and pUC vectors (New England Biolabs, Inc., Ipswich, Mass.)) or expression/purification vectors (such as e.g., GST fusion vectors (Pfizer, Inc., Piscataway, N.J.)) and then expressed in a suitable prokaryotic, viral or eukaryotic host. Purification may then be achieved by conventional means, or in the case of a commercial expression/purification system, in accordance with manufacturer's instructions.

Alternatively, the immunogenic polypeptides of the present disclosure, including variants, may be isolated for example, but without limitation, from wild-type or mutant S. pneumoniae cells, and through chemical synthesization using commercially automated procedures, such as for example, exclusive solid phase synthesis, partial solid phase methods, fragment condensation or solution synthesis.

Polypeptides of the present disclosure preferably have immunogenic activity. “Immunogenic activity” refers to the ability of a polypeptide to elicit an immunological response in a subject. An immunological response to a polypeptide is the development in a subject of a cellular and/or antibody-mediated immune response to the polypeptide. Usually, an immunogical response includes but is not limited to one or more of the following effects: the product of antibodies, B cells, helper T cells, suppressor T cells and/or cytotoxic T cells, directed to an epitope or epitodes of the polypeptide. The term “Epitope” refers to the site on an antigen to which specific B cells and/or T cells respond so that antibody is produced. The immunogenic activity may be protective. The term “Protective immunogenic activity” refers to the ability of a polypeptide to elicit an immunogical response in a subject that prevents or inhibits infection by S. pneumoniae (resulting in disease).

### Compositions

The disclosed immunogenic S. pneumoniae polypeptides are used to produce immunogenic compositions such as, for example, vaccine compositions. An immunogenic composition is one that, upon administration to a subject (e.g., a mammal), induces or enhances an immune response directed against the antigen contained within the composition. This response may include the generation of antibodies (e.g, through the stimulation of B cells) or a T cell-based response (e.g., a cytolytic response). These responses may or may not be protective or neutralizing. A protective or neutralizing immune response is one that is detrimental to the infectious organism corresponding to the antigen (e.g, from which the antigen was derived) and beneficial to the subject (e.g., by reducing or preventing infection). As used herein, protective or neutralizing antibodies may be reactive to the corresponding wild-type S. pneumoniae polypeptide (or fragment thereof) and reduce or inhibit the lethality of the corresponding wild-type S. pneumoniae polypeptide when tested in animals. An immunogenic composition that, upon administration to a host, results in a protective or neutralizing immune response may be considered a vaccine.

The compositions include immunogenic polypeptides in amounts sufficient to elicit an immune response when administered to a subject. Immunogenic compositions used as vaccines comprise an immunogenic polypeptide in an immunologically effective amount, as well as any other components, as needed. By ‘immunologically effective amount’, it is meant that the administration of that amount to a subject, either in a single dose or as part of a series, is effective for treatment or prevention.

In compositions that are comprised of two, three or more immunogenic polypeptides (i.e., PcpA, PhtD, and/or detoxified pneumolysis), the polypeptide components are preferably compatible and are combined in appropriate ratios to avoid antigenic interference and to optimize any possible synergies. For example, the amounts of each component can be in the range of about 5 μg to about 500 μg per dose, 5 μg to about 100 μg per dose; or 10 μg, 25 μg, 50 μg, 75 μg, or 100 μg per dose. Preferably 10 μg, 25 μg or 50 μg of each antigenic/immunogenic component (PcpA, PhtD, and detoxified pneumolysin) is contained in each dose. In one preferred example, a composition includes 25 μg of an immunogenic polypeptide of a Pht polypeptide (“PhtX” such as PhtD), 25 μg of an immunogenic polypeptide of PcpA, and 25 μg of pneumolysin (e.g., SEQ ID NO:10; detoxified pneumolysin such as PlyD1 (SEQ ID NO:9)). In a more preferred example, a composition includes 50 μg of an immunogenic polypeptide of PhtX (e.g., PhtD), 50 μg of an immunogenic polypeptide of PcpA, and 50 μg of pneumolysin (e.g. detoxified pneumolysin such as PlyD1 (SEQ ID NO:9)). A composition comprising immunogenic components of PhtD, PcpA, and detoxified pneumolysin PlyD1 is referred to as “PPrV” in the Examples.

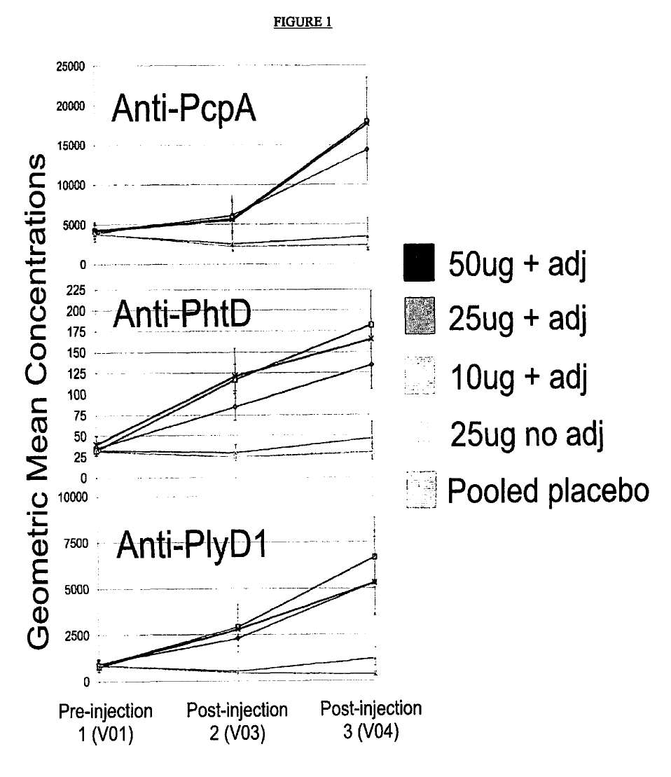

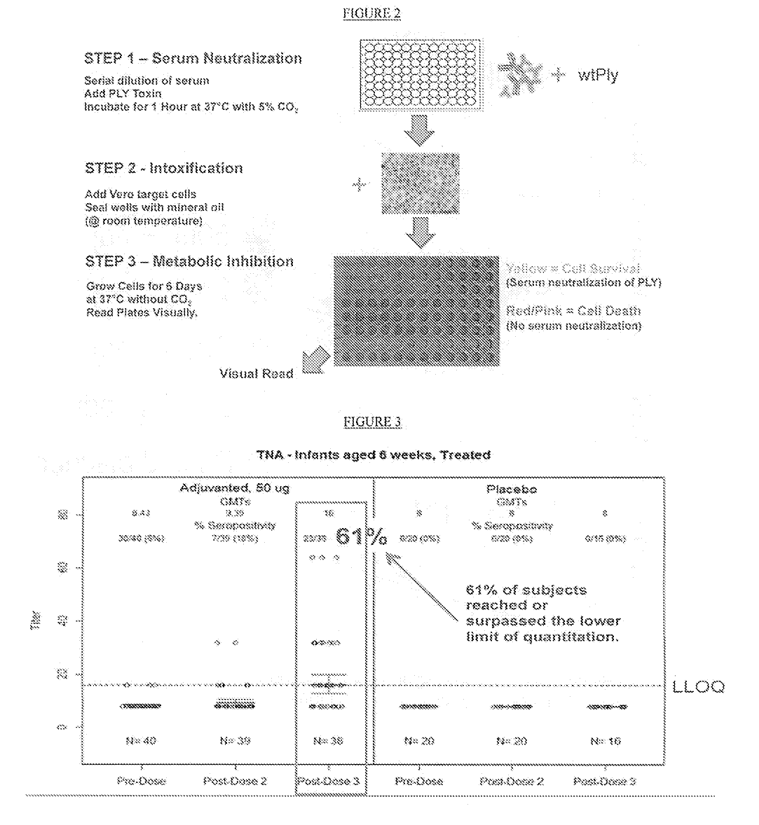

In some embodiments, a particular ratio (weight/weight (w/w)) may provide an enhanced and/or optimal immune response. In certain embodiments, the w/w ratio of PcpA:PhtD:detoxified pneumolysin may be 1:1:1 (e.g., 25 μg each), or 1:1:2 (e.g., 25 μg of each of PcpA and PhtD, with 50 μg detoxified pneumolysin). As shown in the Examples (e.g., FIG. 1), adjuvanted dose formulations having a 1:1:1 ratio (25 μg of each of PcpA, PhtD and the detoxified pneumolysin PlyD1) induced a significant rise in antibodies against PcpA, PhtD and PlyD1 in the infant chort following the second and third administrations. For example, Table 14 shows the results of the post-vaccination infant toxin neutralization assay (FIGS. 2-3). Surprisingly, the data shows that while a significant rise in antibodies was observed for the 25 μg dose in infants (FIG. 1), a significant increased in pneumolysin neutralizing antibodies was observed in the infant population using the 50 μg dose of PlyD1. Thus, it may be necessary to adjust the PcpA:PhtD:PlyD1 ratio to 1:1:2 (w/w), and/or increase the dose of each immunogen at the 1:1:1 ratio (w/w) (e.g., about 50 or 100 μg)) to induce an effective anti-pneumolysin response (e.g., providing pneumolysin neutralization) in certain human subjects (e.g., infants (six to 14 weeks old)). Other variations in these ratios may also be suitable as may be determined by those of ordinary skill in art after consideration of the data described herein.

Compositions of the disclosure can be administered by an appropriate route such as for example, percutaneous (e.g., intramuscular, intravenous, intraperitoneal or subcutaneous), transdermal, mucosal (e.g., intranasal) or topical, in amounts and in regimes determined to be appropriate by those skilled in the art. For example, 1-250 μg or 10-100 μg (e.g., 10, 25, 50 μg) of the immunogens may be administered within a composition. For the purposes of prophylaxis or therapy, the composition can be administered 1, 2, 3, 4 or more times. In one example, the one or more administrations may occur as part of a “prime-boost” protocol. When multiple doses are administered, the doses can be separated from one another by, for example, one week, one month or several months.

Compositions (e.g., vaccine compositions) of the present disclosure may be administered in the presence or absence of an adjuvant. Adjuvants generally are substances that can enhance the immunogenicity of antigens. Adjuvants may play a role in both acquired and innate immunity (e.g., toll-like receptors) and may function in a variety of ways, not all of which are understood.

Many substances, both natural and synthetic, have been shown to function as adjuvants. For example, adjuvants may include, but are not limited to, mineral salts, squalene mixtures, muramyl peptide, saponin derivatives, mycobacterium cell wall preparations, certain emulsions, monophosphoryl lipid A, mycolic acid derivatives, nonionic block copolymer surfactants, Quil A, cholera toxin B subunit, polyphosphazene and derivatives, immunostimulating complexes (ISCOMs), cytokine adjuvants, MF59 adjuvant, lipid adjuvants, mucosal adjuvants, certain bacterial exotoxins and other components, certain oligonucleotides, PLG, and others. These adjuvants may be used in the compositions and methods described herein.

In certain embodiments, the composition is administered in the presence of an adjuvant that comprises an oil-in-water emulsion comprising at least squalene, an aqueous solvent, a polyoxyethylene alkyl ether hydrophilic nonionic surfactant, a hydrophobic nonionic surfactant, wherein said oil-in-water emulsion is obtainable by a phase inversion temperature process and wherein 90% of the population by volume of the oil drops has a size less than 200 nm, and optionally less than 150 nm. Such an adjuvant is described in WO2007006939 (Vaccine Composition Comprising a Thermoinversable Emulsion) which is incorporate herein in its entirety. The composition may also include the product E6020 (having CAS Number 287180-63-6), in addition to, or instead of the described squalene oil-in-water emulsion. Product E6020 is described in US2007/0082875 (which is incorporated herein by reference in its entirety).

In certain embodiments, the composition includes a TLR agonist (e.g., TLR4 agonist) alone or together in combination with an adjuvant. For example, the adjuvant may comprise a TLR4 agonist (e.g., TLA4), squalene, an aqueous solvent, a nonionic hydrophilic surfactant belonging to the polyoxyethylene alkyl ether chemical group, a nonionic hydrophobic surfactant and which is thermoreversible. Examples of such adjuvants are described in WO2007080308 (Thermoreversible Oil-in-Water Emulsion) which is incorporated herein in its entirety. In one embodiment, the composition is adjuvanted with a combination of CpG and an aluminum salt adjuvant (e.g., Alum).

Aluminum salt adjuvants (or compounds) are among the adjuvants of use in the practice of the disclosure. Examples of aluminum salt adjuvants of use include aluminum hydroxide (e.g., crystalline aluminum oxyhydroxide AlO(OH), and aluminum hydroxide Al(OH)₃. Aluminum hydroxide is an aluminum compound comprising Al³⁺ ions and hydroxyl groups (—OH). Mixtures of aluminum hydroxide with other aluminum compounds (e.g., hydroxyphosphate or hydroxysulfate) may also be of use where the resulting mixture is an aluminum compound comprising hydroxyl groups. In particular embodiments, the aluminum adjuvant is aluminum oxyhydroxide (e.g., Alhydrogel®). It is well known in the art that compositions with aluminum salt adjuvants should not be exposed to extreme temperatures, i.e. below freezing (0° C.) or extreme heat (e.g., ≧70° C.) as such exposure may adversely affect the stability and the immunogenicity of both the adsorbed antigen and adjuvant.

The degradation rate of PcpA and PhtD polypeptides when adjuvanted with aluminum hydroxide adjuvant (AlO(OH)) is known to be high (as discussed in the examples below). It was found that adjuvanting PcpA and PhtD polypeptides with an aluminum compound comprising hydroxide groups (e.g., aluminum hydroxide adjuvant) that has been pretreated with phosphate, carbonate, sulfate, carboxylate, diphosphonate or a mixture of two or more of these compounds, increases the stability of these polypeptides. Thus, provided herein are formulations of compositions comprising an immunogenic PcpA polypeptide or an immunogenic PhtX polypeptide (e.g., PhtD) and an aluminum compound comprising hydroxide groups that has been treated with phosphate, carbonate, sulfate, carboxylate, diphosphonate or a mixture of two or more of these compounds, where the treatment increases the stability of the immunogenic polypeptide relative to a composition where the polypeptide is adsorbed to an untreated aluminum compound. In preferred embodiments the aluminum compound is treated with phosphate. Multivalent compositions comprising both immunogenic polypeptides of PcpA and PhtX (e.g., PhtD) and an aluminum compound comprising hydroxide groups that has been treated with phosphate, carbonate, sulfate, carboxylate, diphosphonate or a mixture of two or more of these compounds, where the treatment increases the stability of the immunogenic polypeptides relative to a composition where the polypeptide is adsorbed to an untreated aluminum compound are also provided.

In a particular embodiment of the disclosure, the aluminum compound (e.g., aluminum hydroxide adjuvant) is treated with phosphate, carbonate, sulfate, carboxylate, diphosphonate, or a mixture of two or more of these compounds. By treating the aluminum compound in this way a number of the hydroxyl groups (—OH) in the aluminum compound are replaced with the corresponding ion with which it is being treated (e.g., phosphate (PO₄)). This replacement lowers the PZC of the aluminum compound and the pH of the compound's microenvironment. The phosphate, carbonate, sulfate, carboxylate, or diphosphonate ions are added in an amount sufficient to lower the pH of the microenvironment to a level at which the antigen is stabilized (i.e., the rate of antigen hydrolysis is decreased). The amount necessary will depend on a number of factors such as, for example, the antigen involved, the antigen's isoelectric point, the antigen's concentration, the adjuvanting method utilized, and the amount and nature of any additional antigens present in the formulation. Those skilled in the art in the field of vaccines are capable of assessing the relevant factors and determining the concentration of phosphate, carbonate, sulfate, carboxylate, diphosphonate to add to the aluminum compound to increase the stability of the antigen (and therefore, can prepare the corresponding formulation and composition). For example, titration studies (i.e., adding increasing concentrations of phosphate, etc., to aluminum compound) may be performed.

Phosphate compounds suitable for use include any of the chemical compounds related to phosphoric acid (such as for example, inorganic salts and organic esters of phosphoric acid). Phosphate salts are inorganic compounds containing the phosphate ion (PO₄³⁻), the hydrogen phosphate ion (HPO₄²⁻) or the dihydrogen phosphate ion (H₂PO⁴⁻) along with any cation. Phosphate esters are organic compounds in which the hydrogens of phosphoric acid are replaced by organic groups. Examples of compounds that may be used in place of phosphate salts include anionic amino acids (e.g., glutamate, aspartate) and phospholipids.

Carboxylate compounds suitable for use include any of the organic esters, salts and anions of carboxylic acids (e.g., malic acid, lactic acid, fumaric acid, glutaric acid, EDTA, and EGTA). Sulfer anions suitable for use include any compound containing the sulfate (SO₄ radical) such as salts or esters of sulfuric acid (e.g., sodium sulfate, ammonium sulfate, sulfite, metabisulfite, thiosulfate). Examples of disphosphonate compounds suitable for use include clodronate, pamidronate, tiludronate, and alendronate.

In a preferred embodiment of the disclosure, phosphate is added to aluminum hydroxide adjuvant in the form of a salt. Preferably, the phosphate ions are provided by a buffer solution comprising disodium monosodium phosphate.

In the preferred practice of the present disclosure, as exemplified herein, the aluminum compound (e.g., aluminum oxyhydroxide) is treated with phosphate (for example, by a process as described in the examples). In this process, an aqueous suspension of aluminum oxyhydroxide (approximately 20 mg/mL) is mixed with a phosphate buffer solution (e.g., approximately 400 mol/L). The preferable final phosphate concentration is from about 2 mM to 20 mM. The mixture is then diluted with a buffer (e.g., Tris-HCl, Tris-HCl with saline, HEPES) to prepare a suspension of aluminum oxyhydroxide and phosphate (PO₄). Preferably the buffer is 10 mM Tris-HCl and 150 mM NaCl at a pH of about 7.4. The suspension may then be mixed for approximately 24 hr at room temperature. Preferably the concentration of elemental aluminum in the final suspension is within a range from about 0.28 mg/mL to 1.68 mg/mL. More preferably, the concentration of elemental aluminum is about 0.56 mg/mL.

Immunogenic polypeptides of PcpA, PhtD and detoxified pneumolysis (individually or in combination) may then be adsorbed to the treated aluminum hydroxide. Preferably, approximately 0.2-0.4 mg/mL of antigen is mixed with the suspension of treated aluminum hydroxide adjuvant (e.g., at room temperature or at 2-8° C., in an orbital mixer, for approximately 30 min, or approximately 12-15 hours, or approximately 24 hours).

The percentage of antigen adsorption may be assessed using standard methods known in the art. For example, an aliquot of the antigen/adjuvant preparation may be removed and centrifuged (e.g, at 10,000 rpm) to separate the unadsorbed protein (pellet) from the adjuvant suspension (supernatant). The concentration of protein in the supernatant may be determined using the bicinchoninic acid protein assay (BCA) or reverse phase-high performance liquid chromatography (RP-HPLC). The percentage of adsorption is calculated as follows: % A=100−([PrSN]×100/[PrCtr]) where, [PrSN] is the concentration of protein in supernatant and [PfCtr] is the concentration in the corresponding unadjuvanted control. In preferred embodiments, the % adsorption ranges from about 70% to about 100%. In more preferred embodiments the % adsorption is at least about 70%.

In one embodiment of adjuvanted immunization, immunogenic polypeptides and/or fragments thereof may be covalently coupled to bacterial polysaccharides to form polysaccharide conjugates. Such conjugates may be useful as immunogens for eliciting a T cell dependent immunogenic response directed against the bacterial polysaccharide conjugated to the polypeptides and/or fragments thereof.

The disclosed formulations are stable when stored for prolonged time periods at conventional refrigeration temperatures, e.g., about 2° C. to about 8° C. The formulations exhibit little or no particle agglomeration, no significant decrease in antigen concentration and retain a significant level of immunogenicity and/or antigenicity for at least 6 months or 12 months and preferably for 18 months. The phrase “no significant decrease in antigen concentration” is intended to mean that the composition retains at least 50%, 60%, or 70% of the original antigen concentration, more preferably at least about 80%, 85%, or 90% of the original antigen concentration, more preferably at least about 91%, 92%, 98%, 99% or more of the antigen concentration present when first formulated. Antigen concentration may be measured, for example, by an RP-HPLC, SDS-PAGE or ELISA-based method.

A stable formulation or an immunogenic composition comprising a stable formulation maintains a substantial degree of structural integrity (e.g., maintains a substantial amount of the original antigen concentration, etc.).

Stability may be assessed by measuring for example, the concentration of antigen present (e.g, by RP-HPLC) or by assessing antigen degradation for example by SDS-PAGE analysis. The antigen concentration in the formulation may be compared with that of the formulation as prepared with the same aluminum compound albeit untreated (i.e., not treated with phosphate or carbonate ions). Stability prediction and/or comparison tools include for example, Stability System™ (by ScienTek Software, Inc.), which use Arrhenius Treatment to predict rate constant at storage temperature (2° C.-8° C.). Standard assays for measuring the antigen concentration, and immunogenicity are known in the art and are described in the Examples. Protective efficacy may be assessed by for example evaluating the survival rates of immunized and non-immunized subjects following challenge with a disease causing pathogen or toxin corresponding to the particular antigen present in the formulation.

The immunogenic compositions of the present disclosure are preferably in liquid form, but they may be lyophilized (as per standard methods) or foam dried (as described in US 2009/0110699A1, Antigen-Adjuvant Compositions and Methods). A composition according to one embodiment of the disclosure is in a liquid form. An immunization dose may be formulated in a volume of between 0.5 and 1.0 ml. Liquid formulations may be in any form suitable for administration including for example, a solution, or suspension. Thus, the compositions can include a liquid medium (e.g., saline or water), which may be buffered.

The pH of the formulation (and composition) is preferably between about 6.4 and about 8.4. More preferably, the pH is about 7.4. An exemplary pH range of the compositions is 5-10, e.g., 5-9, 5-8, 5.5-9, 6-7.5, or 6.5-7. The pH may be maintained by the use of a buffer.

The pharmaceutical formulations of the immunogenic compositions of the present disclosure may also optionally include one or more excipients (e.g., diluents, thickeners, buffers, preservatives, surface active agents, adjuvants, detergents and/or immunostimulants) which are well known in the art. Suitable excipients will be compatible with the antigen and with the aluminum adjuvant as is known in the art. Examples of diluents include binder, disintegrants, or dispersants such as starch, cellulose derivatives, phenol, polyethylene glycol, propylene glycol or glycerin. Pharmaceutical formulations may also include one or more active ingredients such as antimicrobial agents, antiinflammatory agents and anesthetics. Examples of detergents include a Tween (polysorbate) such as Tween 80. Suitable excipients for inclusion in the composition of the disclosure are known in the art.

The disclosure provides compositions including PcpA, PhtX (e.g., PhtD) and/or detoxified pneumolysis proteins and one or more pharmaceutically acceptable excipients that provide beneficial properties to the compositions (e.g., increase the stability of one or more of the proteins of the compositions). The compounds or excipients that can be included in the compositions of the disclosure include for example, buffers (e.g., glycine, histidine); tonicity agents (e.g, mannitol); carbohydrates, such as sugars or sugar alcohols (e.g., sorbitol, trehalose, or sucrose; 1-30%) or carbohydrate polymers (e.g., dextran); amino acids, oligopeptides or polyamino acids (up to 100 mM); polyhydric alcohols (e.g., glycerol, and concentrations of up to 20%); detergents, lipids, or surfactants (e.g., Tween 20, Tween 80, or pluronics, with concentrations of up to 0.5%); antioxidants; salts (e.g., sodium chloride, potassium chloride, magnesium chloride, or magnesium acetate, up to 150 mM); or combinations thereof.

In various examples, the excipients may be those that result in increased thermal stability (e.g., of at least 0.5, e.g., 0.5-5, 1-4, or 2-3) as measured by, e.g., the assays described in WO2011/075823 (US Pat. Pub. 2013/0183350 A1).

Exemplary excipients and buffers include sorbitol (e.g., 4-20%, 5-10%), (see Table 11 of WO2011/075823 (US Pat. Pub. 2013/0183350 A1)). These excipients can be used in the disclosure in the concentrations listed in Table 11 of WO2011/075823 (US Pat. Pub. 2013/0183350 A1). Alternatively, the amounts can be varied by, e.g., 0.1-10 fold, as is understood in the art. Other carbohydrates, sugar alcohols, surfactants and amino acids that are known in the art can also be included in the composition of the disclosure.

The excipients and buffers can be used individually or in combination. The pH of such a composition can be, e.g., 5.5-8.0 or 6.5-7.5, and the composition can be stored at, e.g., 2-8° C., in liquid or lyophilized form. In variations of the composition, the sorbitol can be replaced with sucrose (e.g., 4-20%, or 5-10%), or trehalose (e.g., 4-20%, or 5-10%). Other variations of the compositions are included in the disclosure and involve use of other components listed herein. Based on the above, an exemplary composition of the disclosure includes 10% sorbitol, pH 7.4.

In yet a further embodiment, a trivalent formulation composition can include per dose, three proteins (PhtD, PlyD1, PcpA), each in the range of 5 to 50 μg/dose (preferably about 10 μg, 25 μg or 50 μg/dose), and PTH adjuvant (with about 0.56 mg/mL elemental Aluminum containing 2 mM sodium phosphate buffer at about pH 7.5), in about: 10 mM Tris HCl, and about 150 mM NaCl, at about pH 7.4.

In another example, the compositions include sorbitol, or sucrose, which have been shown to provide benefits with respect to stability (see below). The amounts of these components can be, for example, 5-15%, 8-12% or 10% sorbitol or sucrose. A specific example in which these components are present at 10% is described below. In a preferred embodiment the compositions include 10% sorbitol or 10% sucrose.

A composition according to one embodiment of the disclosure may be prepared by (i) treating an aluminum hydroxide adjuvant with phosphate, carbonate, sulfate, carboxylate, diphosphonate or a mixture of two or more of these compounds, and (ii) mixing the treated aluminum hydroxide adjuvant with an immunogenic PcpA polypeptide, an immunogenic PhtX polypeptide and detoxified pneumolysin (e.g., PlyD1). In preferred embodiments, the immunogenic PhtX polypeptide is PhtD.

Immunogenic compositions (e.g. vaccines) containing one or more of the S. pneumoniae polypeptides of the present disclosure may be used to prevent and/or treat S. pneumoniae infections. The prophylactic and therapeutic methods of the disclosure involve vaccination with one or more of the disclosed immunogenic polypeptides in, for example, carrying out the treatment itself, in preventing subsequent infection, or in the production of antibodies for subsequent use in passive immunization.

The immunogenic compositions of the disclosure find use in methods of preventing or treating a disease, disorder, condition or symptoms associated with or resulting from a S. pneumoniae infection The terms disease disorder and condition are used interchangeably herein.

Specifically the prophylactic and therapeutic methods comprise administration of a therapeutically effective amount of a pharmaceutical composition to a subject. In particular embodiments, methods for preventing or treating S. pneumoniae are provided.

As used herein, preventing a disease or disorder is intended to mean administration of a therapeutically effective amount of a pharmaceutical composition of the disclosure to a subject in order to protect the subject from the development of the particular disease or disorder associated with S. pneumoniae.

By treating a disease or disorder is intended administration of a therapeutically effective amount of a pharmaceutical composition of the disclosure to a subject that is afflicted with a disease caused by S. pneumoniae or that has been exposed to S. pneumoniae where the purpose is to cure, heal, alleviate, relieve, alter, remedy, ameliorate, improve, or affect the condition or the symptoms of the disease.

A therapeutically effective amount refers to an amount that provides a therapeutic effect for a given condition and administration regimen. A therapeutically effective amount can be determined by the ordinary skilled medical worker based on patient characteristics (age, weight, gender, condition, complications other diseases etc.). The therapeutically effective amount will be further influenced by the route of administration of the composition.

Also disclosed, is a method of reducing the risk of a pneumococcal disease in a subject comprising administering to the subject an immunogenic composition comprising one or more of the disclosed immunogenic polypeptides. Pneumococcal diseases (i.e., symptomatic infections) include, for example, sinus infection, otitis media, bronchitis, pneumonia, meningitis, hemolytic uremia and bacteremia (septicemia). The risk of any one or more of these infections may be reduced by the methods described herein. Preferred methods include a method of reducing the risk of invasive pneumococcal disease and/or pneumonia in a subject comprising administering to the subject an immunogenic composition comprising an immunogenic PcpA polypeptide, an immunogenic PhtX (e.g., PhtD) polypeptide, and detoxified pneumolysis (e.g., PlyD1).

The present disclosure also provides methods of eliciting an immune response in a mammal by administering the immunogenic compositions, or formulations thereof, to subjects. This may be achieved by the administration of a pharmaceutically acceptable formulation of the compositions to the subject to effect exposure of the immunogenic polypeptide and/or adjuvant to the immune system of the subject. The administrations may occur once or may occur multiple times. In one example, the one or more administrations may occur as part of a so-called “prime-boost” protocol.

Immunogenic compositions may be presented in a kit form comprising the immunogenic composition and an adjuvant or a reconstitution solution comprising one or more pharmaceutically acceptable diluents to facilitate reconstitution of the composition (if in dried form) for administration to a mammal using conventional or other devices. Such a kit would optionally include the device for administration of the liquid form of the composition (e.g. hypodermic syringe, microneedle array) and/or instructions for use.

The compositions and vaccines disclosed herein may also be incorporated into various delivery systems. In one example, the compositions may be applied to a “microneedle array” or “microneedle patch” delivery system for administration. These microneedle arrays or patches generally comprise a plurality of needle-like projections attached to a backing material and coated with a dried form of a vaccine. When applied to the skin of a mammal, the needle-like projections pierce the skin and achieve delivery of the vaccine, effecting immunization of the subject mammal.

Thus, in some embodiments, this disclosure provides compositions comprising an immunogen corresponding to each of PhtD, PcpA, and a detoxified pneumolysin such as PlyD1. In some embodiments, PhtD may have at least about 80% sequence identity to the amino acid sequence as set forth in SEQ ID NO:1, PcpA may have at least about 80% sequence identity to the amino acid sequence as set forth in SEQ ID NO:2, and/or the detoxified pneumolysin may have at least about 80% sequence identity to SEQ ID NO:9 or 10 and/or be a wild-type pneumolysin protein comprising amino acid substitutions at positions 65, 293 and 428 of the wild type sequence (e.g., T₆₅→C, G₂₉₃→C, and C₄₂₈→A as in PlyD1). The immunogenic composition typically induces and/or enhances the production of antibodies having specificity for PcpA, PhtD, and/or detoxified pneumolysin such as PlyD1 upon administration to a host. In some embodiments, the antibodies have specificity for PcpA, PhtD, and detoxified pneumolysin such as PlyD1. The antibodies against detoxified pneumolysin such as PlyD1 may be neutralizing as determined by as assay such as the toxin neutralizing assay (TNA). In some embodiments, the composition may comprise about 10 to about 50 μg of each immuogen, especially about 10 μg, about 25 μg, or about 50 μg. In preferred embodiments, the composition may further comprise at least one adjuvant, especially an aluminum compound (such as e.g., aluminum hydroxide, aluminum phosphate or phosphate treated aluminum hydroxide). In some embodiments, this disclosure provides methods for inducing an immune response against S. pneumoniae in a human being comprising administering to the human being the composition comprising about 10 to about 50 μg (especially about any of 10 μg, 25 μg or 50 μg) of each of PhtD, PcpA, and a detoxified pneumolysin such as PlyD1. In preferred embodiments, the composition may further comprise at least one adjuvant, especially an aluminum compound (such as e.g., aluminum hydroxide, aluminum phosphate or phosphate treated aluminum hydroxide). In some embodiments, the human being may be between about 18 to about 50 years old or older, about 12 to about 13 months old (or older such as about 15 months), or about six to about 14 weeks old (or older). A human infant is typically a human being that is up to about 12 to about 13 months old, such as about six to about 14 weeks old. In some embodiments, the composition may be administered to a human being at least two times (in preferred embodiments, each dose comprising about 25 or about 50 μg of each immuogen with an adjuvant that is preferably aluminum phosphate). In certain embodiments in which the human being is about six to about 14 weeks old, multiple doses of a composition comprising are administered, the initial administration of the composition comprising immunogens corresponding to PhtD, PcpA, and a detoxified pneumolysin (such as PlyD1) may be performed when the human being is about six weeks old. In some such embodiments in which the human being is about six to about 14 weeks old, the mean anti-PcpA geometric mean concentration (GMC) following the second dose is about 5000; the mean anti-PhtD GMC following the second dose is about 100; and the mean anti-detoxified pneumolysin (such as PlyD1) GMC following the second dose is about 2500 (FIG. 1). In preferred embodiments, the composition may be administered to the human being at least three times (in preferred embodiments, each dose comprising about any of 10 μg, 25 μg or 50 μg of each immuogen with an adjuvant that is preferably aluminum phosphate). In some such embodiments in which the human being is about six to about 14 weeks old, the mean anti-PcpA GMC following the third dose is about 15000; the mean anti-PhtD GMC following the third dose is at least about 125; and the mean anti-detoxified pneumolysin (such as PlyD1) GMC following the third dose is about 5000. In some embodiments, the composition is administered to a human being that is about six to about 14 weeks old at least three times, each dose comprises a 1:1:1 (w/w) ratio of each immuogen (e.g., about 50 μg) or each dose comprises 1:1:2 ratio of PcpA, PhtD and detoxified pneumolysin (e.g., 25 μg each of PcpA and PhtD, and about 50 μg of the detoxified pneumolysin), and at least one adjuvant that is preferably an aluminum compound (such as e.g., aluminum hydroxide, aluminum phosphate or phosphate treated aluminum hydroxide), and the human being produces antibodies that neutralize penumolysin after the third dose as determined using a cytotoxicity assay (e.g, as shown in FIGS. 2 and 3). In some embodiments, the methods may induce a neutralizing and/or toxin-neutralizing immune response against S. pneumoniae in a human being. In some such embodiments, the method comprises administering one or more doses of about 25 μg/dose or more of detoxified pneumolysin thereto. In some such embodiments, the composition may also comprise at least one adjuvant, at least one PcpA immunogen, and/or at least one PhtD immunogen.

In some embodiments, the composition may be administered to a human being that is about six to about 14 weeks old (i.e., a human infant) in one, two or three doses (i.e., the composition is administered one, two or three times). The doses are typically separated in time and may also be separated in site and/or route of administration. In some embodiments, the composition may be administered to that human being at least three times and each dose may comprise about 10 μg and/or about 50 μg of each immuogen (e.g., PcpA, PhtD, and a detoxified pneumolysin such as PlyD1). Such compositions may also comprise at least one adjuvant, such as an aluminum compound (e.g., aluminum hydroxide, aluminum phosphate or phosphate treated aluminum hydroxide). In some embodiments, each dose may comprise about 25 μg and/or about 50 μg of each immuogen and at least one adjuvant that is preferably an aluminum compound (e.g., aluminum hydroxide, aluminum phosphate or phosphate treated aluminum hydroxide), and the human being produces antibodies that protect mice from death for at least about 14 days after administering an otherwise lethal dose of S. pneumoniae to the mice (e.g., FIG. 5). In some embodiments, the serum of that human being may be diluted about 1:60 and still protect about 100% of the mice from death (e.g., FIG. 5). In some embodiments, the serum of the human being may be diluted about 1:100 and protect at least about 70% of the mice from death (e.g., FIG. 5).

In some embodiments, methods for inducing and/or enhancing the production of antibodies against PcpA and PhtD, and neutralizing antibodies against a detoxified pneumolysin such as PlyD1 by administering to a human being a composition comprising at least one adjuvant such as an aluminum compound (e.g., aluminum hydroxide, aluminum phosphate or phosphate treated aluminum hydroxide) and immunogens corresponding to PcpA, PhtD, and/or a detoxified pneumolysin, the immunogens being in the composition at a ratio of about 1:1:1 (w/w), are provided. In some preferred embodiments, the human being may be an infant. In some embodiments, the composition(s) used in these methods may comprise about 10 μg of each immunogen. In some embodiments, the composition(s) used in these methods may comprise about 50 μg of each immunogen. In some embodiments, the composition(s) used in these methods may comprise less than about 25 μg of each immunogen. In some embodiments, the composition(s) used in these methods may comprise greater than about 25 μg of each immunogen. In some embodiments, the composition(s) used in these methods may comprise an amount of each immunogen that is not about 25 μg.

In some embodiments, methods for inducing and/or enhancing the production of antibodies against PcpA and PhtD, and/or neutralizing antibodies against a detoxified pneumolysin such as PlyD1, in a human being, the method comprising administering to the infant a composition comprising at least one adjuvant such as an aluminum compound (e.g., aluminum hydroxide, aluminum phosphate or phosphate treated aluminum hydroxide) and immunogens corresponding to PcpA, PhtD, and the detoxified pneumolysin, the immunogens being in the composition at a ratio of about 1:1:2 (w/w). In some embodiments, the composition may also comprise an adjuvant. In some preferred embodiments, the human being may be an infant. In some such embodiments, the composition(s) may comprise about 10 μg of each of immunogens corresponding to PcpA and PhtD. In some such embodiments, the composition(s) may comprise about 50 μg of immunogens corresponding to each of PcpA and PhtD; less than about 25 μg of immunogens corresponding to each of PcpA and PhtD; greater than about 25 μg of of immunogens corresponding to each of PcpA and PhtD; or an amount of each PcpA and PhtD that is not about 25 μg.

In some embodiments, the neutralizing antibodies against pneumolysin are in the serum of the human being, in particular those embodiments in which the human being is an infant. In some embodiments, the neutralizing antibodies may be detected by a toxin neutralization assay (TNA) of the serum of the human being (e.g., infant), with or without cholesterol removal treatment of the serum, performed as is well known in the art. In some embodiments, the composition is administered to the human being in at least one, two or three times. In some embodiments, the neutralizing antibodies against the detoxified pnuemolysin are detected following the first, second, or third administration. In some embodiments, an infant may exhibit an increase geometric mean concentration (GMC) of IgG in serum against each of the immunogens following administration of the composition to the infant once, twice or three times. In some embodiments, an infant may exhibit at least a two-fold rise in IgG antibody concentration in serum against each of the immunogens following administration of the composition to the infant once, twice or three times. In some embodiments, the method comprises administering the composition to an infant at least once, twice or three times and the infant produces antibodies that protect a mouse from death for at least about 14 days after administering an otherwise lethal dose of S. pneumoniae to the mouse. In some embodiments, the serum of the infacnt may be diluted about 1:40 or about 1:80 and protect a mouse from death for at least about 14 days after administering an otherwise lethal dose of S. pneumoniae thereto.

Other embodiments are also contemplated, as would be apparent to those of ordinary skill in the art.

### DEFINITIONS

The term “antigen” as used herein refers to a substance that is capable of initiating and mediating the formation of a corresponding immune body (antibody) when introduced into a mammal or can be bound by a major histocompatibility complex (MHC) and presented to a T-cell. An antigen may possess multiple antigenic determinants such that the exposure of the mammal to an antigen may produce a plurality of corresponding antibodies with differing specificities. Antigens may include, but are not limited to proteins, peptides, polypeptides, nucleic acids and fragments, variants and combinations thereof.

The term “immunogen” is a substance such as a polypeptide or fragment thereof (e.g., a PhtX (e.g., PhtD), PcpA, or detoxified pneumolysis, or an immunogenic fragment thereof) that is able to induce and/or enhance an adaptive immune response (e.g., anti-immunogen antibody production).

The terms peptides, proteins and polypeptides are used interchangeably herein.

An “isolated” polypeptide is one that has been removed from its natural environment. For instance, an isolated polypeptide is a polypeptide that has been removed from the cytoplasm or from the membrane of a cell, and many of the polypeptides, nucleic acids, and other cellular material of its natural environment are no longer present. An “isolatable” polypeptide is a polypeptide that could be isolated from a particular source. A “purified” polypeptide is one that is at least 60% free, preferably at least 75% free, and most preferably at least 90% free from other components with which they are naturally associated. Polypeptides that are produced outside the organism in which they naturally occur, e.g. through chemical or recombinant means, are considered to be isolated and purified by definition, since they were never present in a natural environment.

As used herein, a “fragment” of a polypeptide preferably has at least about 10, 20, 30, 40, 50, 60, 70, 80, 90 or 100 amino acid residues in length. Fragments of S. pneumoniae polypeptides can be generated by methods known to those skilled in the art.

The term “antibody” or “antibodies” includes whole or fragmented antibodies in unpurified or partially purified form (i.e., hybridoma supernatant, ascites, polyclonal antisera) or in purified form. A “purified” antibody is one that is separated from at least about 50% of the proteins with which it is initially found (i.e., as part of a hybridoma supernatant or ascites preparation).

As used in the specification and the appended claims, the singular forms “a”, “an”, and “the” include plural referents unless the context clearly dictates otherwise. Thus, for example, reference to a fragment may include mixtures of fragments and reference to a pharmaceutical carrier or adjuvant may include mixtures of two or more such carriers or adjuvants.

As used herein, a subject or a host is meant to be an individual.

Optional or optionally means that the subsequently described event or circumstance can or cannot occur, and that the description includes instances where the event or circumstance occurs and instances where it does not. For example, the phrase, “optionally the composition can comprise a combination” means that the composition may comprise a combination of different molecules or may not include a combination such that the description includes both the combination and the absence of the combination (i.e., individual members of the combination).

Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint.

When the terms prevent, preventing, and prevention are used herein in connection with a given treatment for a given condition (e.g., preventing S. pneumoniae infection), it is meant to convey that the treated subject either does not develop a clinically observable level of the condition at all, or develops it more slowly and/or to a lesser degree than he/she would have absent the treatment. These terms are not limited solely to a situation in which the subject experiences no aspect of the condition whatsoever. For example, a treatment will be said to have prevented the condition if it is given during exposure of a patient to a stimulus that would have been expected to produce a given manifestation of the condition, and results in the subject's experiencing fewer and/or milder symptoms of the condition than otherwise expected. A treatment can “prevent” infection by resulting in the subject's displaying only mild overt symptoms of the infection; it does not imply that there must have been no penetration of any cell by the infecting microorganism.

Similarly, reduce, reducing, and reduction as used herein in connection with the risk of infection with a given treatment (e.g., reducing the risk of a S. pneumoniae infection) refers to a subject developing an infection more slowly or to a lesser degree as compared to a control or basal level of developing an infection in the absence of a treatment (e.g., administration of an immunogenic polypeptide). A reduction in the risk of infection may result in the subject displaying only mild overt symptoms of the infection or delayed symptoms of infection; it does not imply that there must have been no penetration of any cell by the infecting microorganism.

All references cited within this disclosure are hereby incorporated by reference in their entirety.

### EXAMPLES

The above disclosure generally describes the present disclosure. A more complete understanding can be obtained by reference to the following specific Examples. These Examples are described solely for purposes of illustration and are not intended to limit the scope of the disclosure. Changes in form and substitution of equivalents are contemplated as circumstances may suggest or render expedient. Although specific terms have been employed herein, such terms are intended in a descriptive sense and not for purposes of limitations.

Methods of molecular genetics, protein biochemistry, immunology and fermentation technology used, but not explicitly described in this disclosure and these Examples, are amply reported in the scientific literatures and are well within the ability of those skilled in the art.

### Example 1

### Recombinant PcpA and PhtD Polypeptides

This Example describes the preparation of the PcpA protein and PhtD protein recombinantly. In brief, two recombinantly-derived protein antigens from Streptococcus pneumoniae (strain 14453 (a mouse-virulent capsule serotype 6B strain), deposited on Jun. 27, 1997 as ATCC 55987), PhtD (WO2009/012588) and PcpA (WO 2008/022302) were recombinantly expressed in E. coli, isolated and purified by serial column chromatography following conventional purification protocols.

The phtD gene (but excluding its native signal peptide) was PCR amplified from the S. pneumoniae 14453 genome, using the AccuPrime High Fidelity polymerase (Invitrogen) and primers Spn0211 and Spn0213. Spn0211 and Spn0213 introduced Nod and XhoI restriction sites into the 5′ and 3′ ends, respectively (see Table 2). The PCR product was purified using a QIAquick PCR purification kit (Qiagen) and run on an agarose gene to confirm the size. The PCT product and the pET28a(+) vector (Novagen) were both digested with Ncol and XhoI and subsequently purified from an agarose gel using the QIAEX gel extraction kit (Qiagen). The digested vector and gene were ligated together using T4 DNA ligase (Invitrogen). The ligation mixture was transformed into chemically competent E. coli DH5a and positive clones were selected by plating on Luria agar containing 50 μg/ml kanamycin. DNA from plasmid clone pBAC27 was isolated and was confirmed by sequencing to be correct. The plasmid (pBAC27) was then introduced into E. coli BL21 (DE3) cells by electroporation. Transformed strains were grown at approximately 37° C. and protein expression was induced by the addition of 1 mM IPTG. Expression of gene product was verified by the presence of an induced protein band of the correct size (i.e, approximately 91.9 kDa) by SDS-PAGE analysis.

TABLE 2

| Primer | |
|---|---|
| Name/ | |
| Number | Sequence 5′ → 3′ |
| Spn0211 | CTAGCCATGGGATCCTATGAACTTGGTCGTCACCAAG |
| Spn0213 | AGTCCTCGAGCTACTGTATAGGAGCCCGGTTG |

The predicted amino acid sequence of the polypeptide of pBAC27 is as follows:

(SEQ ID No: 5)
MGSYELGRHQAGQVKKESNRVSYIDGDQAGQKAENLTPDEVSKREGINAE
QIVIKITDQGYVTSHGDHYHYYNGKVPYDAIISEELLMKDPNYQLKDSDI
VNEIKGGYVIKVDGKYYVYLKDAAHADNIRTKEEIKRQKQEHSHNHNSRA
DNAVAAARAQGRYTTDDGYIFNASDIIEDTGDAYIVPHGDHYHYIPKNEL
SASELAAAEAYWNGKQGSRPSSSSSYNANPVQPRLSENHNLTVTPTYHQN
QGENISSLLRELYAKPLSERHVESDGLIFDPAQITSRTARGVAVPHGNHY
HFIPYEQMSELEKRIARIIPLRYRSNHWVPDSRPEQPSPQSTPEPSPSLQ
PAPNPQPAPSNPIDEKLVKEAVRKVGDGYVFEENGVSRYIPAKDLSAETA
AGIDSKLAKQESLSHKLGAKKTDLPSSDREFYNKAYDLLARIHQDLLDNK
GRQVDFEVLDNLLERLKDVSSDKVKLVDDILAFLAPIRHPERLGKPNAQI
TYTDDEIQVAKLAGKYTTEDGYIFDPRDITSDEGDAYVTPHMTHSHWIKK
DSLSEAERAAAQAYAKEKGLTPPSTDHQDSGNTEAKGAEAIYNRVKAAKK
VPLDRMPYNLQYTVEVKNGSLIIPHYDHYHNIKFEWFDEGLYEAPKGYSL
EDLLATVKYYVEHPNERPHSDNGFGNASDHVRKNKADQDSKPDEDKEHDE
VSEPTHPESDEKENHAGLNPSADNLYKPSTDTEETEEEAEDTTDEAEIPQ
VENSVINAKIADAEALLEKVTDPSIRQNAMETLTGLKSSLLLGTKDNNTI
SAEVDSLLALLKESQPAPIQ

The pcpA gene (but excluding the signal sequence and the choline-binding domains) was PCR amplified from the S. pneumoniae 14453 genome using Accuprime Taq DNA polymerase (Invitrogen) and PCR primers (see Table 3) that incorporated restriction endonuclease sites designed for simplified cloning. Plasmid DNA of pET-30a(+) (Novagen) was purified as a low-copy plasmid and prepared for use as the cloning vector by digesting with NdeI and XhoI, followed by gel purification. The resulting 1335 base pair fragment was pcpA (without signal sequence and choline-binding domains) flanked by XhoI (3′-end) and NdeI (5′end) restriction sites. The amplified fragment was cleaned, digested with NdeI and XhoI and then gel purified and ligated into the pET-30a(+) vector. The insert was verified by sequencing and the new plasmid was designated pJMS87.

TABLE 3
(Primers)

| Primer | |
|---|---|
| Name | Sequence 5′ → 3′ |
| UAB 3 | TAGCCTCGAGTTAACCTTTGTCTTTAACCCAACCAACTA |
| | CTCCCTGATTAG |
| UAB- | CTAATGAACCACATATGGCAGATACTCCTAGTTCGGAAG |
| tagless 5 | TAATC |

The predicted amino acid sequence of the polypeptide of pJMS87 is as follows:

(SEQ ID No: 7)
MADTPSSEVIKETKVGSIIQQNNIKYKVLTVEGNIGTVQVGNGVTPVEFE
AGQDGKPFTIPTKITVGDKVFTVTEVASQAFSYYPDETGRIVYYPSSITI
PSSIKKIQKKGFHGSKAKTIIFDKGSQLEKIEDRAFDFSELEEIELPASL
EYIGTSAFSFSQKLKKLTFSSSSKLELISHEAFANLSNLEKLTLPKSVKT
LGSNLFRLTTSLKHVDVEEGNESFASVDGVLFSKDKTQLIYYPSQKNDES
YKTPKETKELASYSFNKNSYLKKLELNEGLEKIGTFAFADAIKLEEISLP
NSLETIERLAFYGNLELKELILPDNVKNFGKHVMNGLPKLKSLTIGNNIN
SLPSFFLSGVLDSLKEIHIKNKSTEFSVKKDTFAIPETVKFYVTSEHIKD
VLKSNLSTSNDIIVEKVDNIKQETDVAKPKKNSNQGVVGWVKDKG

Chemically competent E. coli BL21 (DE3) cells were transformed with plasmid pJMS87 DNA. Expression of gene product was verified by the presence of an induced protein band of the correct size (i.e, approximately 49.4 kDa) by SDS-PAGE analysis.

As the cloned PcpA polypeptide lacks the signal sequence and choline-binding domains, its amino acid sequence correlates with amino acids 27 to 470 of the full length PcpA protein. This region is extremely conserved among all surveyed strains with only 8 variable positions. The most diverged pair of sequences shares 98.7% identity.

The predicted isoelectric points by Vector NTi for the recombinant PcpA protein and the recombinant PhtD protein were 7.19 and 5.16, respectively.

The pcpA gene and phtD gene were each detected in the following serotypes: 1, 2, 3, 4, 5, 6A, 6B, 6C, 7, 7F, 9N, 9V, 11A/B, 11A/D/F, 12F/B, 14, 15B, 15B/C, 16, 18C, 19A, 19F, 22, 23, 23B, 23F, 33F, 34, 35B. A number of these serotypes are not covered by the currently marketed pneumococcal conjugate vaccine PCV7.

The recombinant protein products were expressed, isolated and purified using standard methods.

Adjuvanted monovalent compositions of either recombinant protein were prepared by formulating isolated purified protein with adjuvant (e.g., Aluminum hydroxide adjuvant (e.g. Alhydrogel 85 2%) or AlPO₄) in Tris buffered saline (pH 7.4) using standard methods. Formulated materials were transferred to glass vials and stored at 2° C. to 8° C. Adjuvanted bivalent compositions of both PhtD and PcpA were prepared by aliquoting the desired concentration of each adjuvanted monovalent formulation into a vessel and mixing on a nutator for approximately 0.5 hours at room temperature. Desired formulation volumes were then aliquoted into sterile 3 mL glass vials with rubber stopper closure and aluminum cap. Alternatively, bivalent compositions were prepared by mixing the desired concentration of each isolated purified protein together and then formulating mixture with adjuvant in Tris buffered saline (pH 7.4).

Detoxified pneumolysis (PlyD1) (SEQ ID NO.: 9) was prepared using standard techniques as has been described in the art (e.g., U.S. Pat. Pub. No. 2011/0287046A1).

### Example 2

This Example describes the preparation of a surface modified adjuvant and formulations with this adjuvant. A surface modified adjuvant was prepared by treating aluminum hydroxide adjuvant (Alhydrogel™, Brenntag) with phosphate. The aluminum hydroxide adjuvant used was a wet gel suspension which according to the manufacturer tolerates re-autoclavation but is destroyed if frozen. According to the manufacturer, when the pH is maintained at 5-7, the adjuvant has a positive charge and can adsorb negatively charged antigens (e.g., proteins with acidic isoelectric points when kept at neutral pH).
a) Phosphate treatment of AlO(OH)—An aqueous suspension of AlO(OH) (approximately 20 mg/mL) was mixed with a stock solution of phosphate buffer (approximately 400 mol/L) and diluted with 10 mM Tris-HCL buffer (Sigma Aldrich) at about pH 7.4 to prepare a phosphate-treated AlO(OH) suspension (herein referred to as “PTH”) having approximately 13 mg/mL AlOOH/200 mM PO4. This suspension was then mixed for approximately 30 minutes to 24 hr at room temperature.
b) Antigen adsorption—Recombinantly-derived PcpA, PhtD and PlyD1 antigens (expressed, isolated and purified as described in Example 1A) were individually adsorbed to the phosphate-treated AlO(OH).

A mixture was prepared containing about 0.2-0.4 mg/mL of purified antigen (i.e., rPcpA, rPhtD and PlyD1) each antigen and 0.56 mg elemental aluminum/ml/PO₄ mM of the PTH suspension. Alternatively, mixtures were prepared containing purified antigen with aluminum hydroxide adjuvant (as Alhydrogel® 85 2%) or AlPO₄ in Tris buffered saline (pH 7.4) using standard methods. The mixtures whereas mixed in an orbital shaker for about 30 minutes to 24 hours at room temperature to facilitate the association of antigen and adjuvant. Similar adsorptions were prepared a number of times and the typical pre-adsorbed composition was: protein (PhtD or PcpA): 0.2-0.4 mg/ml, phosphate: 2 to 20 80 mM (preferably, 2 to 20 mM) and AlO(OH): 1.25 mg/ml (0.56 mg of elemental Al/ml). Prepared antigen adsorbed samples were stored at about 2° C.-8° C. until used. Alternatively, antigens were adjuvanted together (to prepare trivalent formulations) by using a stock solution of phosphate treated aluminum hydroxide adjuvant.
c) Preparation of a trivalent formulation (PPrV vaccine)—The intermediate bulk lots (monovalent formulations) of PhtD adsorbed to PTH, PcpA adsorbed to PTH and PlyD1 adsorbed to PTH were blended and mixed together for about 30 minutes at room temperature in an orbital shaker to prepare a trivalent formulation.

### Example 3

This example describes the administration of the trivalent formulation (Example 2C) (PPrV vaccine) in healthy adults (18-50 years), toddlers (12-13 months) and infant (6 weeks) human beings in a randomized, placebo-controlled, observer-blind step-down study. Adults and toddlers received one injection of 50 μg (each antigen, adjuvanted (“high dose” (“HD”)), 60 subjects) or placebo (10 subjects). Infants received three injections (three doses) of 10 μg (each antigen, adjuvanted (“low dose” (“LD”), 40 subjects), 25 μg (each antigen, adjuvanted (“medium dose” (“MD”), 40 subjects), or 50 μg (each antigen, adjuvanted (“high dose” (“HD”), 40 subjects) or placebo (20 subjects) at six, ten and 14 weeks of age who also received concomitant standard-of-care vaccines. Study readouts included standard safety measures, ELISA (anti-PcpA, anti-PhtD, and anti-PlyD1 IgG), toxin neutralization assay (functional assay for PlyD1), passive protection (functional assay), and nasopharyngeal carriage. The vaccine was found to be well tolerated by all age groups (adults, toddlers and infants): no protocol-defined threshold criteria were met; no reports of immediate adverse effects (AEs) or analphylaxis; no immediate AEs, AEs, or SAEs leading to study discontinuation; no related SAEs, no life-threatening SAEs and no deaths. As illustrated by FIG. 1, all adjuvanted dose formulations induced a significant rise in antibodies against PcpA, PhtD and PlyD1 in the infant chort following the second and third administrations of the trivalent vaccine. The unadjuvanted 25 μg group was not considered more efficacious than placebo; more specifically, the unadjuvanted group did not produce a response significantly greater than the placebo group. Tables 4-14 provide additional data regarding administration of a composition comprising the PcpA, PhtD and PlyD1 immunogens (“PPrV”) in infants. For instance, the GMC ratios in infants were statistically significant for total anti-PcpA IgG post-injection 3 based on the pre-Injection 1 response and post-Injection 3 based on the post-injection 2 response following the adjuvanted 10 μg, 25 μg and 50 μg PPrV injections (Table 6). The GMC ratios in infants were also statistically significant for total anti-PhtD and anti-PlyD1 IgG, respectively, for each of the injection comparisons following the adjuvanted 10 μg, 25 μg and 50 μg PPrV injections (Tables 7, 8). In the adjuvanted 10 μg, 25 μg and 50 μg PPrV groups, a ≧2 fold-rise in IgG antibody concentration against PcpA, PhtD, and PlyD1 was observed in the majority of infants (Table 9). And the percentage of infants with a ≧2 fold-rise or a a ≧4 fold-rise of IgG against the antigens increased following each subsequent administration (Table 9). All were higher than in the 25 μg undajuvanted group. The adjuvant effect is further illustrated in Table 11 with respect to GMCs.

TABLE 4
GMCs in Infants

| | | PPrV 10 μg + adj | | | PPrV 25 μg + adj | | | PPrV 25 μg | | |
|---|---|---|---|---|---|---|---|---|---|---|
| | | (N = 28) | | | (N = 32) | | | (N = 31) | | |
| | | M | GMC | (95% CI) | M | GMC | (95% CI) | M | GMC | (95% CI) |
| anti-PcpA IgG | Pre-Injection 1 (V01) | 28 | 3938 | (2989; 5189) | 32 | 4284 | (3611; 5083) | 31 | 3780 | (2887; 4950) |
| (ELISA-EU/mL) | Post-Injection 2 (V03) | 28 | 6134 | (4332; 8685) | 32 | 5728 | (4190; 7830) | 31 | 2548 | (1632; 3976) |
| | Post-Injection 3 (V04) | 28 | 14347 | (10386; 19818) | 32 | 17928 | (14520; 22135) | 31 | 3450 | (2057; 5785) |
| anti-PhtD IgG | Pre-Injection 1 (V01) | 28 | 35.0 | (29.3; 41.9) | 32 | 32.6 | (26.9; 39.6) | 31 | 32.8 | (27.0; 39.7) |
| (ELISA-EU/mL) | Post-Injection 2 (V03) | 28 | 84.4 | (68.3; 104) | 32 | 117 | (101; 135) | 31 | 29.5 | (22.4; 39.0) |
| | Post-Injection 3 (V04) | 28 | 134 | (105; 170) | 32 | 182 | (153; 216) | 31 | 47.1 | (33.3; 66.5) |
| anti-PlyD1 IgG | Pre-Injection 1 (V01) | 28 | 945 | (736; 1213) | 32 | 842 | (632; 1122) | 31 | 844 | (641; 1110) |
| (ELISA-EU/mL) | Post-Injection 2 (V03) | 28 | 2309 | (1557; 3422) | 32 | 2909 | (2191; 3864) | 31 | 528 | (393; 709) |
| | Post-Injection 3 (V04) | 28 | 5322 | (3510; 8071) | 32 | 6667 | (5036; 8826) | 31 | 1197 | (794; 1806) |

| | | PPrV 50 μg + adj | | | Placebo-Pooled | | |
|---|---|---|---|---|---|---|---|
| | | (N = 33) | | | (N = 47) | | |
| | | M | GMC | (95% CI) | M | GMC | (95% CI) |
| anti-PepA IgG | Pre-Injection 1 (V01) | 33 | 4190 | (3309; 5306) | 47 | 3832 | (3209; 4576) |
| (ELISA-EU/mL) | Post-Injection 2 (V03) | 33 | 5581 | (3878; 8032) | 47 | 2222 | (1771; 2789) |
| | Post-Injection 3 (V04) | 33 | 17633 | (13221; 23518) | 47 | 2347 | (1716; 3210) |
| anti-PhtD IgG | Pre-Injection 1 (V01) | 33 | 39.9 | (31.9; 49.8) | 47 | 31.5 | (26.7; 37.1) |
| (ELISA-EU/mL) | Post-Injection 2 (V03) | 33 | 121 | (94.6; 154) | 47 | 24.6 | (19.7; 30.7) |
| | Post-Injection 3 (V04) | 33 | 165 | (121; 223) | 47 | 30.0 | (22.8; 39.5) |
| anti-PlyD1 IgG | Pre-Injection 1 (V01) | 33 | 789 | (527; 1182) | 47 | 869 | (662; 1141) |
| (ELISA-EU/mL) | Post-Injection 2 (V03) | 33 | 2800 | (1890; 4146) | 47 | 441 | (327; 596) |
| | Post-Injection 3 (V04) | 33 | 5286 | (3609; 7743) | 47 | 345 | (254; 470) |

Source Data:
N: number of subjects analyzed according to the per-protocol analysis set.
M: number of subjects available for the endpoint.
The 2-sided 95% CI of a geometric mean is based on the Student t-distribution.

TABLE 5
Comparison of GMCs in Infants (Total anti-PcpA, by visit and vaccine group)

| | | PPrV | PPrV | PPrV | PPrV | Placebo- |
|---|---|---|---|---|---|---|
| | | 10 μg + adj | 25 μg + adj | 25 μg | 50 μg + adj | Pooled |
| | | (N = 28) | (N = 32) | (N = 31) | (N = 33) | (N = 47) |
| Post-Injection 2 | M | 28 | 32 | 35 | 33 | 47 |
| response based on | Geometric Mean Ratio | 1.56 | 1.34 | 0.674 | 1.33 | 0.580 |
| pre-Injection 1 | (95% CI) | (0.976; 2.49) | (0.922; 1.94) | (0.466; 0.975) | (0.921; 1.93) | (0.441; 0.762) |
| response | p-value | 0.0624 | 0.1216 | 0.0370 | 0.1239 | 0.0002 |
| Post-Injection 3 | M | 28 | 32 | 31 | 33 | 47 |
| response based on | Geometric Mean Ratio | 3.64 | 4.18 | 0.913 | 4.21 | 0.612 |
| pre-Injection 1 | (95% CI) | (2.30; 5.77) | (3.14; 5.58) | (0.556; 1.50) | (2.97; 5.96) | (0.414; 0.906) |
| response | p-value | <.0001 | <.0001 | 0.7090 | <.0001 | 0.0153 |
| Post-Injection 3 | M | 28 | 32 | 31 | 33 | 47 |
| response based on | Geometric Mean Ratio | 2.34 | 3.13 | 1.35 | 3.16 | 1.06 |
| post-Injection 2 | (95% CI) | (1.86; 2.94) | (2.48; 3.95) | (1.07; 1.72) | (2.47; 4.04) | (0.854; 1.31) |
| response | p-value | <.0001 | <.0001 | 0.0150 | <.0001 | 0.6087 |

Source Data: [symbol]
N: number of subjects analyzed according to the per-protocol analysis set.
M: number of subjects available for the endpoint.
GMFR is the geometric mean of the individual concentration ratios of post-dose result over pre-dose result.
The 2-sided 95% CI of a geometric mean is based on the Student t-distribution.
The p-value of the geometric mean ratio is based on the paired t-test. [symbol]
indicates data missing or illegible when filed

TABLE 6
Comparison of GMCs in Infants (Total anti-PhtD, by visit and vaccine group)

| | | PPrV | PPrV | PPrV | PPrV | Placebo- |
|---|---|---|---|---|---|---|
| | | 10 μg + adj | 25 μg + adj | 25 μg | 50 μg + adj | Pooled |
| | | (N = 28) | (N = 32) | (N = 31) | (N = 33) | (N = 47) |
| Post-Injection 2 | M | 28 | 32 | 31 | 33 | 47 |
| response based on | Geometric Mean Ratio | 2.41 | 3.57 | 0.902 | 3.02 | 0.782 |
| pre-Injection 1 | (95% CI) | (1.83; 3.17) | (2.78; 4.60) | (0.664; 1.23) | (2.17; 4.22) | (0.601; 1.02) |
| response | p-value | <.0001 | <.0001 | 0.4977 | <.0001 | 0.0655 |
| Post-Injection 3 | M | 28 | 32 | 31 | 33 | 47 |
| response based on | Geometric Mean Ratio | 3.81 | 5.57 | 1.44 | 4.13 | 0.953 |
| pre-Injection 1 | (95% CI) | (2.86; 5.09) | (4.41; 7.03) | (0.974; 2.12) | (2.63; 6.47) | (0.688; 1.32) |
| response | p-value | <.0001 | <.0001 | 0.0664 | <.0001 | 0.7688 |
| Post-Injection 3 | M | 28 | 32 | 31 | 33 | 47 |
| response based on | Geometric Mean Ratio | 1.58 | 1.56 | 1.59 | 1.36 | 1.22 |
| post-Injection 2 | (95% CI) | (1.32; 1.91) | (1.35; 1.80) | (1.23; 2.06) | (1.05; 1.78) | (1.02; 1.46) |
| response | p-value | <.0001 | <.0001 | 0.0009 | 0.0230 | 0.0327 |

Source Data: [symbol]
N: number of subjects analyzed according to the per-protocol analysis set.
M: number of subjects available for the endpoint.
GMFR is the geometric mean of the individual concentration ratios of post-dose result over pre-dose result.
The 2-sided 95% CI of a geometric mean is based on the Student t-distribution.
The p-value of the geometric mean ratio is based on the paired t-test. [symbol]
indicates data missing or illegible when filed

TABLE 7
Comparison of GMCs in Infants (Total anti-PlyD1, by visit and vaccine group)

| | | PPrV | PPrV | PPrV | PPrV | Placebo- |
|---|---|---|---|---|---|---|
| | | 10 μg + adj | 25 μg + adj | 25 μg | 50 μg + adj | Pooled |
| | | (N = 28) | (N = 32) | (N = 31) | (N = 33) | (N = 47) |
| Post-Injection 2 | M | 28 | 32 | 31 | 33 | 47 |
| response based | Geometric Mean Ratio | 2.44 | 3.46 | 0.626 | 3.55 | 0.507 |
| on pre-Injection | (95% CI) | (1.53; 3.90) | (2.32; 5.15) | (0.435; 0.902) | (2.03; 6.19) | (0.398; 0.647) |
| 1 response | p-value | 0.0005 | <.0001 | 0.0136 | <.0001 | <.0001 |
| Post-Injection 3 | M | 28 | 32 | 31 | 33 | 47 |
| response based | Geometric Mean Ratio | 5.63 | 7.92 | 1.42 | 6.70 | 0.397 |
| on pre-Injection | (95% CI) | (3.28; 9.68) | (5.06; 12.4) | (0.869; 2.32) | (4.08; 11.0) | (0.305; 0.518) |
| 1 response | p-value | <.0001 | <.0001 | 0.1558 | <.0001 | <.0001 |
| Post-Injection 3 | M | 28 | 32 | 31 | 33 | 47 |
| response based | Geometric Mean Ratio | 2.31 | 2.29 | 2.27 | 1.89 | 0.783 |
| on post-Injection | (95% CI) | (1.46; 3.64) | (1.77; 2.96) | (1.56; 3.30) | (1.22; 2.93) | (0.592; 1.03) |
| 2 response | p-value | 0.0009 | <.0001 | 0.0001 | 0.0058 | 0.0837 |

Source Data: [symbol]
N: number of subjects analyzed according to the per-protocol analysis set.
M: number of subjects available for the endpoint.
GMFR is the geometric mean of the individual concentration ratios of post-dose result over pre-dose result.
The 2-sided 95% CI of a geometric mean is based on the Student t-distribution.
The p-value of the geometric mean ratio is based on the paired t-test. [symbol]
indicates data missing or illegible when filed

TABLE 8
Summary of Fold-Rise Response (Antibodies) in Infants

| | | | PPrV 10 μg + adj | | | PPrV 25 μg + adj | | | PPrV 25 μg | | |
|---|---|---|---|---|---|---|---|---|---|---|---|
| | | | (N = 28) | | | (N = 32) | | | (N = 31) | | |
| | | | n/M | % | (95% CI) | n/M | % | (95% CI) | n/M | % | (95% CI) |
| anti-PcpA IgG | Post-Injection 2/ | ≧2 fold-rise | 9/28 | 32.1 | (15.9; 52.4) | 10/32 | 31.3 | (16.1; 50.0) | 7/31 | 22.6 | (9.6; 41.1) |
| | pre-Injection 1 | ≧4 fold-rise | 7/28 | 25.0 | (10.7; 44.9) | 6/32 | 18.8 | (7.2; 36.4) | 2/31 | 6.5 | (0.8; 21.4) |
| | Post-Injection 3/ | ≧2 fold-rise | 21/28 | 75.0 | (55.1; 89.3) | 25/32 | 78.1 | (60.0; 90.7) | 10/31 | 32.3 | (16.7; 51.4) |
| | pre-Injection 1 | ≧4 fold-rise | 12/28 | 42.9 | (24.5; 62.8) | 18/32 | 56.3 | (37.7; 73.6) | 5/31 | 16.1 | (5.5; 33.7) |
| | Post-Injection 3/ | ≧2 fold-rise | 18/28 | 64.3 | [symbol] 4.1; 81.4) | 25/32 | 78.1 | (60.0; 90.7) | 6/31 | 19.4 | (7.5; 37.5) |
| | post-Injection 2 | ≧4 fold-rise | 3/28 | 10.7 | (2.3; 28.2) | 8/32 | 25.0 | (11.5; 43.4) | 2/31 | 6.5 | (0.8; 21.4) |
| anti-PhtD IgG | Post-Injection 2/ | ≧2 fold-rise | 18/28 | 64.3 | (44.1; 81.4) | 26/32 | 81.3 | (63.6; 92.8) | 6/31 | 19.4 | (7.5; 37.5) |
| | pre-Injection 1 | ≧4 fold-rise | 6/28 | 21.4 | (8.3; 41.0) | 16/32 | 50.0 | (31.9; 68.1) | 1/31 | 3.2 | (0.1; 16.7) |
| | Post-Injection 3/ | ≧2 fold-rise | 23/28 | 82.1 | (63.1; 93.9) | 29/32 | 90.6 | (75.0; 98.0) | 11/31 | 35.5 | (19.2; 54.6) |
| | pre-Injection 1 | ≧4 fold-rise | 16/28 | 57.1 | (37.2; 75.5) | 23/32 | 71.9 | (53.3; 86.3) | 6/31 | 19.4 | (7.5; 37.5) |
| | Post-Injection 3/ | ≧2 fold-rise | 9/28 | 32.1 | (15.9; 52.4) | 10/32 | 31.3 | (16.1; 50.0) | 12/31 | 38.7 | (21.8; 57.8) |
| | post-Injection 2 | ≧4 fold-rise | 1/28 | 3.6 | (0.1; 18.3) | 1/32 | 3.1 | (0.1; 16.2) | 3/31 | 9.7 | (2.0; 25.8) |
| anti-PlyD1 | Post-Injection 2/ | ≧2 fold-rise | 13/28 | 46.4 | (27.5; 66.1) | 21/32 | 65.6 | (46.8; 81.4) | 4/31 | 12.9 | (3.6; 29.8) |
| IgG | pre-Injection 1 | ≧4 fold-rise | 9/28 | 32.1 | (15.9; 52.4) | 16/32 | 50.0 | (31.9; 68.1) | 1/31 | 3.2 | (0.1; 16.7) |
| | Post-Injection 3/ | ≧2 fold-rise | 22/28 | 78.6 | (59.0; 91.7) | 29/32 | 90.6 | (75.0; 98.0) | 12/31 | 38.7 | (21.8; 57.8) |
| | pre-Injection 1 | ≧4 fold-rise | 19/28 | 67.9 | (47.6; 84.1) | 23/32 | 71.9 | (53.3; 86.3) | 7/31 | 22.6 | (9.6; 41.1) |
| | Post-Injection 3/ | ≧2 fold-rise | 20/28 | 71.4 | (51.3; 86.8) | 20/32 | 62.5 | (43.7; 78.9) | 15/31 | 48.4 | (30.2; 66.9) |
| | post-Injection 2 | ≧4 fold-rise | 10/28 | 35.7 | (18.6; 55.9) | 5/32 | 15.6 | (5.3; 32.8) | 10/31 | 32.3 | (16.7; 51.4) |

| | | | PPrV 50 μg + adj | | | Placebo Pooled | | |
|---|---|---|---|---|---|---|---|---|
| | | | (N = 33) | | | (N = 47) | | |
| | | | n/M | % | (95% CI) | n/M | % | (95% CI) |
| anti-PcpA IgG | Post-Injection 2/ | ≧2 fold-rise | 12/33 | 36.4 | (20.4; 54.9) | 4/47 | 8.5 | (2.4; 20.4) |
| | pre-Injection 1 | ≧4 fold-rise | 6/33 | 18.2 | (7.0; 35.5) | 2/47 | 4.3 | (0.5; 14.5) |
| | Post-Injection 3/ | ≧2 fold-rise | 25/33 | 75.8 | (57.7; 88.9) | 13/47 | 27.7 | (15.6; 42.6) |
| | pre-Injection 1 | ≧4 fold-rise | 15/33 | 45.5 | (28.1; 63.6) | 3/47 | 6.4 | (1.3; 17.5) |
| | Post-Injection 3/ | ≧2 fold-rise | 25/33 | 75.8 | [symbol] 7.7; 88.9) | 9/47 | 19.1 | (9.1; 33.3) |
| | post-Injection 2 | ≧4 fold-rise | 12/33 | 36.4 | (20.4; 54.9) | 4/47 | 8.5 | (2.4; 20.4) |
| anti-PhtD IgG | Post-Injection 2/ | ≧2 fold-rise | 20/33 | 60.6 | (42.1; 77.1) | 6/47 | 12.8 | (4.8; 25.7) |
| | pre-Injection 1 | ≧4 fold-rise | 12/33 | 36.4 | (20.4; 54.9) | 4/47 | 8.5 | (2.4; 20.4) |
| | Post-Injection 3/ | ≧2 fold-rise | 25/33 | 75.8 | (57.7; 88.9) | 11/47 | 23.4 | (12.3; 38.0) |
| | pre-Injection 1 | ≧4 fold-rise | 21/33 | 63.6 | (45.1; 79.6) | 7/47 | 14.9 | (6.2; 28.3) |
| | Post-Injection 3/ | ≧2 fold-rise | 9/33 | 27.3 | (13.3; 45.5) | 8/47 | 17.0 | (7.6; 30.8) |
| | post-Injection 2 | ≧4 fold-rise | 1/33 | 3.0 | (0.1; 15.8) | 1/47 | 2.1 | (0.1; 11.3) |
| anti-PlyD1 | Post-Injection 2/ | ≧2 fold-rise | 19/33 | 57.6 | (39.2; 74.5) | 2/47 | 4.3 | (0.5; 14.5) |
| IgG | pre-Injection 1 | ≧4 fold-rise | 12/33 | 36.4 | (20.4; 54.9) | 2/47 | 4.3 | (0.5; 14.5) |
| | Post-Injection 3/ | ≧2 fold-rise | 27/33 | 81.8 | (64.5; 93.0) | 3/47 | 6.4 | (1.3; 17.5) |
| | pre-Injection 1 | ≧4 fold-rise | 23/33 | 69.7 | (51.3; 84.4) | 1/47 | 2.1 | (0.1; 11.3) |
| | Post-Injection 3/ | ≧2 fold-rise | 21/33 | 63.6 | (45.5; 79.6) | 6/47 | 12.8 | (4.8; 25.7) |
| | post-Injection 2 | ≧4 fold-rise | 8/33 | 24.2 | (11.1; 42.3) | 1/47 | 2.1 | (0.1; 11.3) |

Source Data: [symbol]
N: number of subjects analyzed according to the per-protocol analysis set.
M: number of subjects available for the endpoint.
Exact 2-sided 95% CI for the single proportion is based on the Clopper-Pearson method. [symbol]
indicates data missing or illegible when filed

TABLE 9
Dose Effect Comparisons in Infants, by Observed GMCs

| | | PPrV 10 μg + adj/ | | | PPrV 25 μg + adj/ | | |
|---|---|---|---|---|---|---|---|
| | | Placebo-Pooled | | | Placebo-Pooled | | |
| | | GMC | | | GMC | | |
| | | Ratio | (95% CI) | p-value | Ratio | (95% CI) | p-value |
| anti-PcpA IgG | Pre-Injection 1 (V01) | 1.03 | (0.756; 1.40) | 0.8595 | 1.12 | (0.868; 1.44) | 0.3832 |
| | Post-Injection 2 (V03) | 2.76 | (1.87; 4.08) | <.0001 | 2.58 | (1.78; 3.73) | <.0001 |
| | Post-Injection 3 (V04) | 6.11 | (3.82; 9.78) | <.0001 | 7.64 | (5.06; 11.5) | <.0001 |
| anti-PhtD IgG | Pre-Injection 1 (V01) | 1.11 | (0.867; 1.43) | 0.3973 | 1.04 | (0.807; 1.33) | 0.7785 |
| | Post-Injection 2 (V03) | 3.43 | (2.48; 4.75) | <.0001 | 4.74 | (3.55; 6.33) | <.0001 |
| | Post-Injection 3 (V04) | 4.45 | (3.00; 6.61) | <.0001 | 6.05 | (4.23; 8.66) | <.0001 |
| anti-PlyD1 IgG | Pre-Injection 1 (V01) | 1.09 | (0.731; 1.62) | 0.6494 | 0.968 | (0.649; 1.44) | 0.8736 |
| | Post-Injection 2 (V03) | 5.23 | (3.22; 8.51) | <.0001 | 6.59 | (4.30; 10.1) | <.0001 |
| | Post-Injection 3 (V04) | 15.4 | (9.31; 25.5) | <.0001 | 19.3 | (12.5; 29.8) | <.0001 |

| | |
|---|---|
| PPrV 50 μg + adj/ | PPrV 25 μg + adj/ |
| Placebo-Pooled | PPrV 10 μg + adj |

| | | GMC | | | GMC | | |
|---|---|---|---|---|---|---|---|
| | | Ratio | (95% CI) | p-value | Ratio | (95% CI) | p-value |
| anti-PcpA IgG | Pre-Injection 1 (V01) | 1.09 | (0.822; 1.45) | 0.5338 | 1.09 | (0.799; 1.48) | 0.5974 |
| | Post-Injection 2 (V03) | 2.51 | (1.68; 3.75) | <.0001 | 0.934 | (0.592; 1.47) | 0.7652 |
| | Post-Injection 3 (V04) | 7.51 | (4.84; 11.7) | <.0001 | 1.25 | (0.864; 1.81) | 0.2309 |
| anti-PhtD IgG | Pre-Injection 1 (V01) | 1.27 | (0.971; 1.65) | 0.0808 | 0.931 | (0.718; 1.21) | 0.5875 |
| | Post-Injection 2 (V03) | 4.90 | (3.53; 6.80) | <.0001 | 1.38 | (1.08; 1.76) | 0.0104 |
| | Post-Injection 3 (V04) | 5.48 | (3.64; 8.24) | <.0001 | 1.36 | (1.02; 1.81) | 0.0346 |
| anti-PlyD1 IgG | Pre-Injection 1 (V01) | 0.907 | (0.572; 1.44) | 0.6760 | 0.891 | (0.611; 1.30) | 0.5442 |
| | Post-Injection 2 (V03) | 6.35 | (3.93; 10.2) | <.0001 | 1.26 | (0.790; 2.01) | 0.3250 |
| | Post-Injection 3 (V04) | 15.3 | (9.47; 24.7) | <.0001 | 1.25 | (0.775; 2.03) | 0.3518 |

| | | PPrV 50 μg + adj/ | | | PPrV 50 μg + adj/ | | |
|---|---|---|---|---|---|---|---|
| | | PPrV 10 μg + adj | | | PPrV 25 μg + adj | | |
| | | GMC | | | GMC | | |
| | | Ratio | (95% CI) | p-value | Ratio | (95% CI) | p-value |
| anti-PcpA IgG | Pre-Injection 1 (V01) | 1.06 | (0.747; 1.51) | 0.7264 | 0.978 | (0.734; 1.30) | 0.8779 |
| | Post-Injection 2 (V03) | 0.910 | (0.553; 1.50) | 0.7059 | 0.974 | (0.608; 1.56) | 0.9126 |
| | Post-Injection 3 (V04) | 1.23 | (0.806; 1.88) | 0.3326 | 0.984 | (0.692; 1.40) | 0.9254 |
| anti-PhtD IgG | Pre-Injection 1 (V01) | 1.14 | (0.854; 1.52) | 0.3706 | 1.22 | (0.914; 1.63) | 0.1720 |
| | Post-Injection 2 (V03) | 1.43 | (1.04; 1.97) | 0.0299 | 1.03 | (0.783; 1.37) | 0.8088 |
| | Post-Injection 3 (V04) | 1.23 | (0.835; 1.81) | 0.2877 | 0.906 | (0.641; 1.28) | 0.5667 |
| anti-PlyD1 IgG | Pre-Injection 1 (V01) | 0.835 | (0.513; 1.36) | 0.4423 | 0.937 | (0.575; 1.53) | 0.7899 |
| | Post-Injection 2 (V03) | 1.21 | (0.701; 2.10) | 0.4843 | 0.962 | (0.597; 1.55) | 0.8725 |
| | Post-Injection 3 (V04) | 0.993 | (0.571; 1.73) | 0.9805 | 0.793 | (0.497; 1.26) | 0.3242 |

TABLE 10
Adjuvant Effect Comparison in Infants

| | | PPrV 25 μg + adj/ | | | PPrV 25 μg/ | | | PPrV 25 μg + adj/ | | |
|---|---|---|---|---|---|---|---|---|---|---|
| | | Placebo-Pooled | | | Placebo-Pooled | | | PPrV 25 μg | | |
| | | GMC | | | GMC | | | GMC | | |
| | | Ratio | (95% CI) | p-value | Ratio | (95% CI) | p-value | Ratio | (95% CI) | p-value |
| anti- | Pre-Injection 1 (V01) | 1.12 | (0.868; 1.44) | 0.3832 | 0.987 | (0.728; 1.34) | 0.9297 | 1.13 | (0.831; 1.55) | 0.4270 |
| PcpA | Post-Injection 2 (V03) | 2.58 | (1.78; 3.73) | <.0001 | 1.15 | (0.733; 1.79) | 0.5807 | 2.25 | (1.32; 3.82) | 0.0033 |
| IgG | Post-Injection 3 (V04) | 7.64 | (5.06; 11.5) | <.0001 | 1.47 | (0.840; 2.57) | 0.1743 | 5.20 | (3.03; 8.92) | <.0001 |
| anti- | Pre-Injection 1 (V01) | 1.04 | (0.807; 1.33) | 0.7785 | 1.04 | (0.809; 1.34) | 0.7570 | 0.996 | (0.763; 1.30) | 0.9785 |
| PhtD | Post-Injection 2 (V03) | 4.74 | (3.55; 6.33) | <.0001 | 1.20 | (0.848; 1.70) | 0.2985 | 3.95 | (2.92; 5.34) | <.0001 |
| IgG | Post-Injection 3 (V04) | 6.05 | (4.23; 8.66) | <.0001 | 1.57 | (1.02; 2.41) | 0.0418 | 3.86 | (2.65; 5.63) | <.0001 |
| anti- | Pre-Injection 1 (V01) | 0.968 | (0.649; 1.44) | 0.8736 | 0.970 | (0.653; 1.44) | 0.8809 | 0.998 | (0.676; 1.47) | 0.9914 |
| PlyD1 | Post-Injection 2 (V03) | 6.59 | (4.30; 10.1) | <.0001 | 1.20 | (0.775; 1.85) | 0.4126 | 5.51 | (3.69; 8.22) | <.0001 |
| IgG | Post-Injection 3 (V04) | 19.3 | (12.5; 29.8) | <.0001 | 3.47 | (2.11; 5.70) | <.0001 | 5.57 | (3.43; 9.04) | <.0001 |

Source Data: [symbol]
N: number of subjects analyzed according to the per-protocol analysis set.
M: number of subjects available for the endpoint.
The 2-sided 95% CI of a GMC and GMC ratio is based on the Student t-distribution. [symbol]
indicates data missing or illegible when filed

Functional activity of the PlyD1 immunogen was assessed from serum treated to remove cholesterol in an in vitro toxin neutralization assay (TNA) against the wild-type pneumolysin protein. In some assays, cholesterol was removed from the test serum as it may compete in the assay. A summary of the GMTs is provided in Tables 11-13. Table 14 also summarizes the results of the post-vaccination infant toxin neutralization assay (FIGS. 2-3). Surprisingly, while a significant rise in antibodies was observed for the 25 μg dose in infants (FIG. 1), a significant increase in PlyD1 neutralizing antibodies was only observed in the infant population using the 10 μg (as compared to pre-vaccination) or 50 μg dose of PlyD1 (as compared to pre-vaccination, 10 μg dose, or 25 μg dose) (Table 4). Thus, it may be necessary to adjust the PcpA:PhtD:PlyD1 ratio to 1:1:2 (w/w), or increase the dose of each antigen to 50 μg (e.g., maintaining the 1:1:1 ratio (w/w)) to induce an effective anti-PlyD1 response.

TABLE 11
Summary of Observed Geometric Means of Titers (GMTs in Infants) - TNA Analysis

| | | PPrV 10 μg + adj | | | PPrV 25 μg + adj | | | PPrV 25 μg | | |
|---|---|---|---|---|---|---|---|---|---|---|
| | | (N = 40) | | | (N = 40) | | | (N = 40) | | |
| | | M | GMT | (95% CI) | M | GMT | (95% CI) | M | GMT | (95% CI) |
| TNA | Pre-Injection 1 (V01) | 40 | 8.43 | (7.80; 9.11) | 40 | 8.57 | (8.02; 9.17) | 40 | 8.43 | (7.94; 8.94) |
| antibodies | Post-Injection 2 (V03) | 40 | 9.68 | (8.56; 10.9) | 39 | 8.90 | (8.08; 9.81) | 38 | 8.15 | (7.85; 8.45) |
| | Post-Injection 3 (V04) | 37 | 12.5 | (10.3; 15.3) | 39 | 11.8 | (9.99; 14.0) | 38 | 8.76 | (8.11; 9.48) |

| | | PPrV 50 μg + adj | | | Pooled Placebo | | |
|---|---|---|---|---|---|---|---|
| | | (N = 40) | | | (N = 60) | | |
| | | M | GMT | (95% CI) | M | GMT | (95% CI) |
| TNA | Pre-Injection 1 (V01) | 40 | 8.43 | (7.94; 8.94) | 60 | 8.09 | (7.91; 8.28) |
| antibodies | Post-Injection 2 (V03) | 39 | 9.39 | (8.32; 10.6) | 58 | 8.00 | (NC) |
| | Post-Injection 3 (V04) | 38 | 16.0 | (12.8; 20.0) | 53 | 8.00 | (NC) |

Source Data: [symbol]
N: number of subjects analyzed according to the TNA analysis set 1.
M: number of subjects available for the endpoint.
The 2-sided 95% CI of a geometric mean is based on the Student t-distribution. [symbol]
indicates data missing or illegible when filed

TABLE 13
Summary of Fold-Rise Response in TNA Antibodies in Infants

| | | | PPrV 10 μg + adj | | | PPrV 25 μg + adj | | | PPrV 25 μg | | |
|---|---|---|---|---|---|---|---|---|---|---|---|
| | | | (N = 40) | | | (N = 40) | | | (N = 40) | | |
| | | | n/M | % | (95% CI) | n/M | % | (95% CI) | n/M | % | (95% CI) |
| TNA antibodies | Post-Injection 2/pre-Injection 1 | ≧2 fold-rise | 2/40 | 5.0 | (0.6; 16.9) | 1/39 | 2.6 | (0.1; 13.5) | 0/38 | 0.0 | (0.0; 9.3) |
| | | ≧4 fold-rise | 0/40 | 0.0 | (0.0; 8.8) | 0/39 | 0.0 | (0.0; 9.0) | 0/38 | 0.0 | (0.0; 9.3) |
| | Post-Injection 3/pre-Injection 1 | ≧2 fold-rise | 7/37 | 18.9 | (8.0; 35.2) | 4/39 | 10.3 | (2.9; 24.2) | 0/38 | 0.0 | (0.0; 9.3) |
| | | ≧4 fold-rise | 1/37 | 2.7 | (0.1; 14.2) | 1/39 | 2.6 | (0.1; 13.5) | 0/38 | 0.0 | (0.0; 9.3) |
| | Post-Injection 3/post-Injection 2 | ≧2 fold-rise | 7/37 | 18.9 | (8.0; 35.2) | 4/39 | 10.3 | (2.9; 24.2) | 0/38 | 0.0 | (0.0; 9.3) |
| | | ≧4 fold-rise | 1/37 | 2.7 | (0.1; 14.2) | 1/39 | 2.6 | (0.1; 13.5) | 0/38 | 0.0 | (0.0; 9.3) |

| | | | PPrV 50 μg + adj | | | Pooled Placebo | | |
|---|---|---|---|---|---|---|---|---|
| | | | (N = 40) | | | (N = 60) | | |
| | | | n/M | % | (95% CI) | n/M | % | (95% CI) |
| TNA antibodies | Post-Injection 2/pre-Injection 1 | ≧2 fold-rise | 2/39 | 5.1 | (0.6; 17.3) | 0/58 | 0.0 | (0.0; 6.2) |
| | | ≧4 fold-rise | 0/39 | 0.0 | (0.0; 9.0) | 0/58 | 0.0 | (0.0; 6.2) |
| | Post-Injection 3/pre-Injection 1 | ≧2 fold-rise | 12/38 | 31.6 | (17.5; 48.7) | 0/53 | 0.0 | (0.0; 6.7) |
| | | ≧4 fold-rise | 3/38 | 7.9 | (1.7; 21.4) | 0/53 | 0.0 | (0.0; 6.7) |
| | Post-Injection 3/post-Injection 2 | ≧2 fold-rise | 11/38 | 28.9 | (15.4; 45.9) | 0/53 | 0.0 | (0.0; 6.7) |
| | | ≧4 fold-rise | 2/38 | 5.3 | (0.6; 17.7) | 0/53 | 0.0 | (0.0; 6.7) |

Source Data: [symbol]
N: number of subjects analyzed according to the TNA analysis set 1.
M: number of subjects available for the endpoint.
Exact two-sided 95% CI for the single proportion is based on the Clopper-Pearson method. [symbol]
indicates data missing or illegible when filed

TABLE 14

| | | Percent ≧ Lower Limit | |
|---|---|---|---|
| | | of Quantitation^{†} (LLOQ) | |
| | | Treated (cholesterol | Untreated (cholesterol |
| | | depleted) | intact) |
| 10 μg | N = 37 | *43% | 78% |
| Placebo 10 μg | N = 18 | 0% | 11% |
| 25 μg | N = 39 | 44%^{‡} | 77% |
| Placebo 25 μg | N = 19 | 0% | 11% |
| 50 μg | N = 38 | *61%^{‡} | 95% |
| Placebo 50 μg | N = 16 | 0% | 56% |

^{†}Lower Limit of Quantitation = 13.3
*Significant increase from prevaccination versus postvaccination TNA antibodies GMTs for the 10 μg group (p = 0.03) and 50 μg group (p < 0.001)
^{‡}At post dose 3, all adjuvanted dose groups had significantly higher TNA antibodies GMTs compared to pooled placebo GMTs. The post dose 3, GMTs for the 50 ug group were also significantly higher than the 25 ug group.

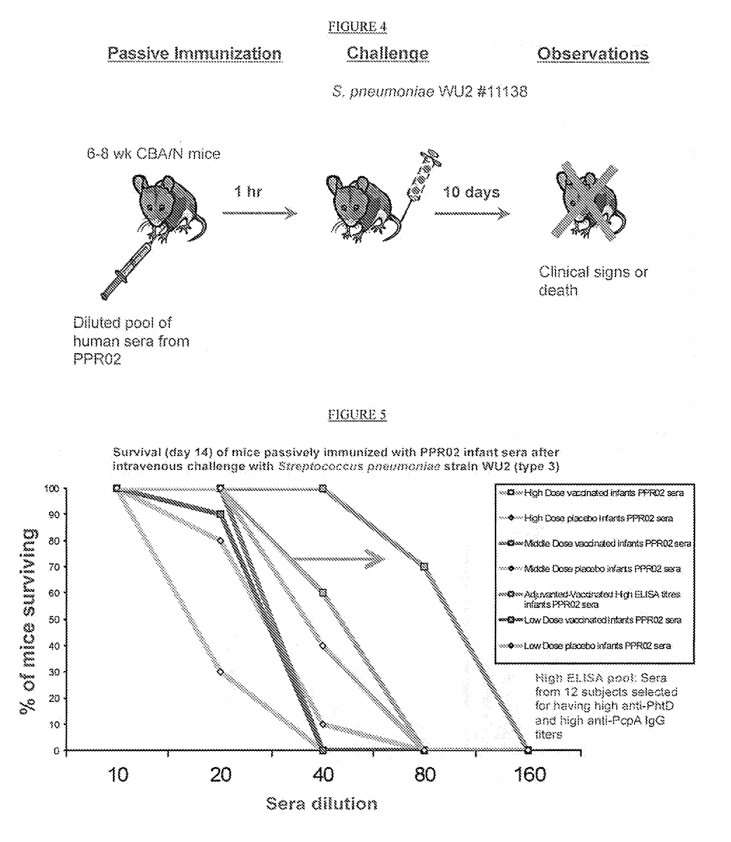

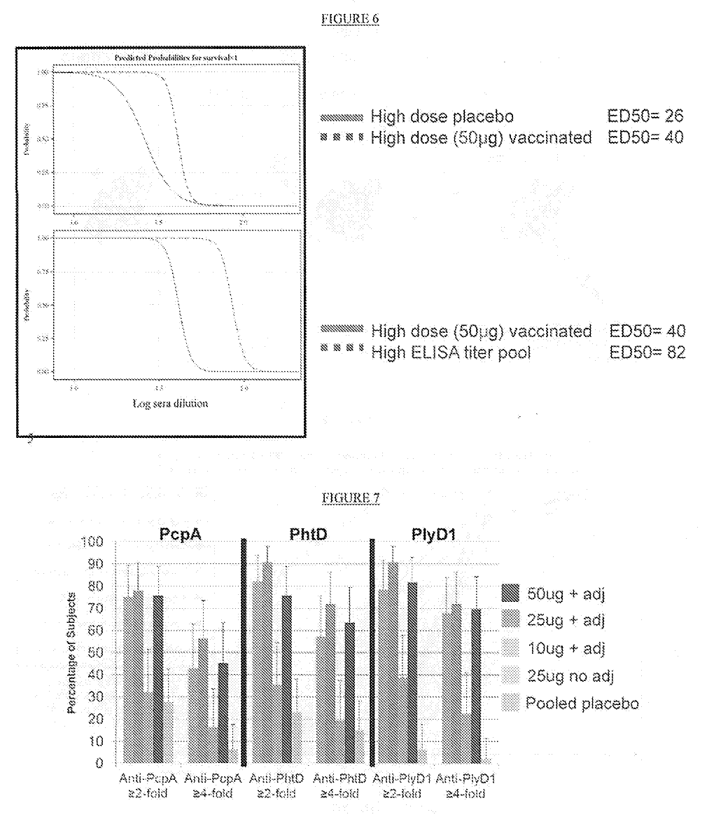

It was also observed in the passive immunization studies (FIG. 4) that serum having higher ELISA titers (anti-PhtD and anti-PcpA IgG) conferred better protection to intravenous challenge with Streptococcus pneumoniae in passively immunized mice (see, e.g., the “Adjuvanted-Vaccinated High ELISA titres infants PPR02 sera” group) (FIGS. 5, 6). PlyD1 and PhtD were found to produce the highest proportion of participants exhibiting two- and four-fold responses (FIG. 7).

### REFERENCES

1. Henrichsen J. Six newly recognized types of Streptococcus pneumoniae.
2. Park I H, Pritchard D G, Cartee R et al. 2007. Discovery of a new capsular serotype (6C) within serogroup 6 of Streptococcus pneumoniae. J. Clin. Microbiol. 45, 1225-1233.
3. World Health Organization. 2007. Pneumococcal conjugate vaccine for childhood immunization—WHO position paper. Wkly Epidemiol. Rec. 82, 93-104.
4. Plotkin, S. A. and Orenstein W. A. Vaccines. Editors W. B. Saunders Company, Third Edition 1999
5. Fedson, D. S. et al, (1999), The burden of pneumococcal disease among adults in developed and developing countries: what is known and what is not known. Vaccine 17, S11-S18.
6. Klein, D. L. (1999) Pneumococcal disease and the role of conjugate vaccines. Microb. Drug Resist., 5, 147-157.
7. Rahav, G., et al, (1997) Invasive pneumococcal infection: A comparison between adults and children. Medicine 76, 295:303.
8. World Health Organization Bulletin 2004. Global estimate of the incidence of clinical pneumonia among children under five years of age. December 2004, 82 (12).
9. Siber G R, Klugman K P, Makela P H. Pneumococcal Vaccines: The Impact of Conjugate Vaccine. Washington D.C.: ASM Press; 2008
10. PREVNAR® (package insert). Wyeth Pharmaceuticals Inc. Philadelphia, Pa. 2006
11. Clinical and Vaccine Immunology, June 2007, p. 792-795; Pediatr. Infect. Dis. J. 16(4 Suppl.):S97-S102.
12. WHO (2005). Guidelines on nonclinical evaluations vaccines. Technical report series No. 927.

1-33. (canceled)

34. A method for inducing antibodies against PcpA, PhtD and detoxified pneumolysin, in a human infant, the method comprising administering to the infant a composition comprising at least one adjuvant and immunogens corresponding to PcpA, PhtD, and detoxified pneumolysin, the immunogens being in the composition at a ratio of about 1:1:1, PcpA:PhtD:detoxified pneumolysin (w/w).

35. The method of claim 34 wherein the composition comprises less than about 25 μg of each immunogen, about 10 μg of each immunogen, greater than about 25 μg of each immunogen, or about 50 of each immunogen.

36. The method of claim 34 wherein the composition comprises less than about 25 μg of immunogens corresponding to each of PcpA and PhtD, about 10 μg of immunogens corresponding to each of PcpA and Phil), greater than about 25 μg of immunogens corresponding to each of PcpA and PhtD, or about 50 μg of immunogens corresponding to each of PcpA and PhtD.

37. The method of claim 34 wherein the infant is about 12 to about 13 months old.

38. The method of claim 34 wherein the infant is between about six to about 14 weeks old.

39. The method of claim 34 wherein the composition is administered to the infant at least one, two or three times.

40. The method of claim 39 wherein the composition is administered at least two or three times at an interval between doses of about 4 weeks.

41. The method of claim 39 wherein the first administration of the composition is performed when the human being is between about six to about 14 weeks old or between about 12 to about 13 months old.

42. The method of claim 34 wherein the infant exhibits an increase geometric mean concentration (GMC) of IgG in serum against each of the immunogens following administration of the composition to the infant once, twice or three times.

43. The method of claim 34 wherein the infant exhibits at least at two-fold rise in IgG antibody concentration in serum against each of the immunogens following administration of the composition to the infant once, twice or three times.

44. The method of claim 34 wherein neutralizing antibodies are induced against detoxified pneumolysin.

45. The method of claim 44 wherein the neutralizing antibodies are in the serum of the infant.

46. The method of claim 45 wherein the neutralizing antibodies are detected following the first, second, or third administration.

47. The method of claim 34 wherein the composition is administered to the infant at least once, twice or three times and the infant produces antibodies that protect a mouse from death for at least about 14 days after administering an otherwise lethal dose of S. pneumoniae to the mouse.

48. The method of claim 47 wherein the serum of the human being may be diluted about 1:40 or about 1:80 and protect a mouse from death for at least about 14 days after administering an otherwise lethal dose of S. pneumoniae thereto.

49. The method of claim 34 wherein the at least one adjuvant is an aluminum compound.

50. The method of claim 49 wherein the aluminum compound is aluminum hydroxide.

51. The method of claim 34 wherein wherein PhtD has at least 80% sequence identity to the amino acid sequence as set forth in SEQ II) NO:1; PcpA has at least 80% sequence identity to the amino acid sequence as set forth in SEQ ID NO:2.

52. The method of claim 34 wherein the modified pneumolysin has at least 80% sequence identity to the amino acid sequence of SEQ ID NO.:9 and includes at least cysteine at amino acid 65, cysteine at amino acid 293 and alanine at amino acid 428.

53. A method for inducing antibodies against PcpA, Phil) and detoxified pneumolysin, in a human infant, the method comprising administering to the infant a composition comprising at least one adjuvant and immunogens corresponding to PcpA, PhtD, and a detoxified pneumolysin, the immunogens being in the composition at a ratio of about 1:1:2, PcpA:PhtD:detoxified pneumolysin (w/w).

54. The method of claim 53 wherein the composition comprises less than about 25 μg of an immunogen corresponding to detoxified pneumolysin, about 10 tag of an immunogen corresponding to detoxified pneumolysin, greater than about 25 μg of an immunogen corresponding to detoxified pneumolysin, or about 50 μs of an immunogen corresponding to detoxified pneumolysin.

55. The method of claim 53 neutralizing antibodies are induced against detoxified pneumolysin.

56. The method of any one of claim 53 wherein the neutralizing antibodies are in the serum of the infant.

57. The method of claim 54 wherein the neutralizing antibodies are detected by a toxin neutralization assay of the serum of the infant, with or without cholesterol removal treatment of the serum.

58. A method for inducing a toxin-neutralizing immune response against S. pneumoniae in a human being by administering one or more doses of about 25 μg/dose or more of detoxified pneumolysin thereto.

59. The method of claim 58 further comprising at least one adjuvant, at least one PcpA immunogen, and/or at least one PhtD immunogen.


SEQUENCE LISTING (14 sequences)

SEQ ID NO: 1 - protein, 838 aa, Streptococcus pneumoniae
MKINKKYLAGSVAVLALSVCSYELGRHQAGQVKKESNRVSYIDGDQAGQKAENLTPDEVSKREGINAEQIVIKITDQGYVTSHGDHYHYYNGKVPYDAIISEELLMKDPNYQLKDSDIVNEIKGGYVIKVDGKYYVYLKDAAHADNIRTKEEIKRQKQEHSHNHNSRADNAVAAARAQGRYTTDDGYIFNASDIIEDTGDAYIVPHGDHYHYIPKNELSASELAAAEAYWNGKQGSRPSSSSSYNANPVQPRLSENHNLTVTPTYHQNQGENISSLLRELYAKPLSERHVESDGLIFDPAQITSRTARGVAVPHGNHYHFIPYEQMSELEKRIARIIPLRYRSNHWVPDSRPEQPSPQSTPEPSPSLQPAPNPQPAPSNPIDEKLVKEAVRKVGDGYVFEENGVSRYIPAKDLSAETAAGIDSKLAKQESLSHKLGAKKTDLPSSDREFYNKAYDLLARIHQDLLDNKGRQVDFEVLDNLLERLKDVSSDKVKLVDDILAFLAPIRHPERLGKPNAQITYTDDEIQVAKLAGKYTTEDGYIFDPRDITSDEGDAYVTPHMTHSHWIKKDSLSEAERAAAQAYAKEKGLTPPSTDHQDSGNTEAKGAEAIYNRVKAAKKVPLDRMPYNLQYTVEVKNGSLIIPHYDHYHNIKFEWFDEGLYEAPKGYSLEDLLATVKYYVEHPNERPHSDNGFGNASDHVRKNKADQDSKPDEDKEHDEVSEPTHPESDEKENHAGLNPSADNLYKPSTDTEETEEEAEDTTDEAEIPQVENSVINAKIADAEALLEKVTDPSIRQNAMETLTGLKSSLLLGTKDNNTISAEVDSLLALLKESQPAPIQ

SEQ ID NO: 2 - protein, 641 aa, Streptococcus pneumoniae
MKKTTILSLTTAAVILAAYVPNEPILADTPSSEVIKETKVGSIIQQNNIKYKVLTVEGNIRTVQVGNGVTPVEFEAGQDGKPFTIPTKITVGDKVFTVTEVASQAFSYYPDETGRIVYYPSSITIPSSIKKIQKKGFHGSKAKTIIFDKGSQLEKIEDRAFDFSELEEIELPASLEYIGTSAFSFSQKLKKLTFSSSSKLELISHEAFANLSNLEKLTLPKSVKTLGSNLFRLTTSLKHVDVEEGNESFASVDGVLFSKDKTQLIYYPSQKNDESYKTPKETKELASYSFNKNSYLKKLELNEGLEKIGTFAFADAIKLEEISLPNSLETIERLAFYGNLELKELILPDNVKNFGKHVMNGLPKLKSLTIGNNINSLPSFFLSGVLDSLKEIHIKNKSTEFSVKKDTFAIPETVKFYVTSEHIKDVLKSNLSTSNDIIVEKVDNIKQETDVAKPKKNSNQGVVGWVKDKGLWYYLNESGSMATGWVKDKGLWYYLNESGSMATGWVKDKGLWYYLNESGSMATGWVKDKGLWYYLNESGSMATGWVKDKGLWYYLNESGSMATGWVKDKGLWYYLNESGSMATGWVKDKGLWYYLNESGSMATGWFTVSGKWYYTYNSGDLLVNTTTPDGYRVNANGEWVG

SEQ ID NO: 3 - DNA, 2514 bp, Streptococcus pneumoniae
atgaaaatcaataaaaaatatctagcaggttcagtggcagtccttgccctaagtgtttgttcctatgaacttggtcgtcaccaagctggtcaggttaagaaagagtctaatcgagtttcttatatagatggtgatcaggctggtcaaaaggcagaaaatttgacaccagatgaagtcagtaagagagaggggatcaacgccgaacaaattgttatcaagattacggatcaaggttatgtgacctctcatggagaccattatcattactataatggcaaggttccttatgatgccatcatcagtgaagaacttctcatgaaagatccgaattatcagttgaaggattcagacattgtcaatgaaatcaagggtggctatgtgattaaggtagacggaaaatactatgtttaccttaaagatgcggcccatgcggacaatattcggacaaaagaagagattaaacgtcagaagcaggaacacagtcataatcataactcaagagcagataatgctgttgctgcagccagagcccaaggacgttatacaacggatgatgggtatatcttcaatgcatctgatatcattgaggacacgggtgatgcttatatcgttcctcacggcgaccattaccattacattcctaagaatgagttatcagctagcgagttagctgctgcagaagcctattggaatgggaagcagggatctcgtccttcttcaagttctagttataatgcaaatccagttcaaccaagattgtcagagaaccacaatctgactgtcactccaacttatcatcaaaatcaaggggaaaacatttcaagccttttacgtgaattgtatgctaaacccttatcagaacgccatgtagaatctgatggccttattttcgacccagcgcaaatcacaagtcgaaccgccagaggtgtagctgtccctcatggtaaccattaccactttatcccttatgaacaaatgtctgaattggaaaaacgaattgctcgtattattccccttcgttatcgttcaaaccattgggtaccagattcaagaccagaacaaccaagtccacaatcgactccggaacctagtccaagtctgcaacctgcaccaaatcctcaaccagctccaagcaatccaattgatgagaaattggtcaaagaagctgttcgaaaagtaggcgatggttatgtctttgaggagaatggagtttctcgttatatcccagccaaggatctttcagcagaaacagcagcaggcattgatagcaaactggccaagcaggaaagtttatctcataagctaggagctaagaaaactgacctcccatctagtgatcgagaattttacaataaggcttatgacttactagcaagaattcaccaagatttacttgataataaaggtcgacaagttgattttgaggttttggataacctgttggaacgactcaaggatgtctcaagtgataaagtcaagttagtggatgatattcttgccttcttagctccgattcgtcatccagaacgtttaggaaaaccaaatgcgcaaattacctacactgatgatgagattcaagtagccaagttggcaggcaagtacacaacagaagacggttatatctttgatcctcgtgatataaccagtgatgagggggatgcctatgtaactccacatatgacccatagccactggattaaaaaagatagtttgtctgaagctgagagagcggcagcccaggcttatgctaaagagaaaggtttgacccctccttcgacagaccatcaggattcaggaaatactgaggcaaaaggagcagaagctatctacaaccgcgtgaaagcagctaagaaggtgccacttgatcgtatgccttacaatcttcaatatactgtagaagtcaaaaacggtagtttaatcatacctcattatgaccattaccataacatcaaatttgagtggtttgacgaaggcctttatgaggcacctaaggggtatagtcttgaggatcttttggcgactgtcaagtactatgtcgaacatccaaacgaacgtccgcattcagataatggttttggtaacgctagtgaccatgttcgtaaaaataaggcagaccaagatagtaaacctgatgaagataaggaacatgatgaagtaagtgagccaactcaccctgaatctgatgaaaaagagaatcacgctggtttaaatccttcagcagataatctttataaaccaagcactgatacggaagagacagaggaagaagctgaagataccacagatgaggctgaaattcctcaagtagagaattctgttattaacgctaagatagcagatgcggaggccttgctagaaaaagtaacagatcctagtattagacaaaatgctatggagacattgactggtctaaaaagtagtcttcttctcggaacgaaagataataacactatttcagcagaagtagatagtctcttggctttgttaaaagaaagtcaaccggctcctatacag

SEQ ID NO: 4 - DNA, 1923 bp, Streptococcus pneumoniae
atgaaaaaaactacaatattatcattaactacagctgcggttattttagcagcatatgtccctaatgaaccaatcctagcagatactcctagttcggaagtaatcaaagagactaaagttggaagtattattcaacaaaataatatcaaatataaggttctaactgtagaaggtaacataagaactgttcaagtgggtaatggagttactcctgtagagtttgaagctggtcaagatggaaaaccattcacgattcctacaaaaatcacagtaggtgataaagtatttaccgttactgaagtagctagtcaagcttttagttattatccagatgaaacaggtagaattgtctactatcctagctctattactatcccatcaagcataaaaaaaatacaaaaaaaaggcttccatggaagtaaagctaaaactattatttttgacaaaggcagtcagctggagaaaattgaagatagagcttttgatttttctgaattagaagagattgaattgcctgcatctctagaatatattggaacaagtgcattttcttttagtcaaaaattgaaaaagctaaccttttcctcaagttcaaaattagaattaatatcacatgaggcttttgctaatttatcaaatttagagaaactaacattaccaaaatcggttaaaacattaggaagtaatctatttagactcactactagcttaaaacatgttgatgttgaagaaggaaatgaatcgtttgcctcagttgatggtgttttgttttcaaaagataaaactcaattaatttattatccaagtcaaaaaaatgacgaaagttataaaacgcctaaggagacaaaagaacttgcatcatattcgtttaataaaaattcttacttgaaaaaactcgaattgaatgaaggtttagaaaaaatcggtacttttgcatttgcggatgcgattaaacttgaagaaattagcttaccaaatagtttagaaactattgaacgtttagccttttacggtaatttagaattaaaagaacttatattaccagataatgttaaaaattttggtaaacacgttatgaacggtttaccaaaattaaaaagtttaacaattggtaataatatcaactcattgccgtccttcttcctaagtggcgtcttagattcattaaaggaaattcatattaagaataaaagtacagagttttctgtgaaaaaagatacatttgcaattcctgaaactgttaagttctatgtaacatcagaacatataaaagatgttcttaaatcaaatttatctactagtaatgatatcattgttgaaaaagtagataatataaaacaagaaactgatgtagctaaacctaaaaagaattctaatcagggagtagttggttgggttaaagacaaaggtttatggtattacttaaacgaatcaggttcaatggctactggttgggttaaagacaaaggtttatggtattacttaaacgaatcaggttcaatggctactggttgggttaaagacaaaggcttatggtattacttaaacgaatcaggttcaatggctactggttgggttaaagacaaaggcttatggtattacttaaatgaatcaggttcaatggctactggttgggttaaagacaaaggcttatggtattacttaaacgaatcaggttcaatggctactggttgggttaaagacaaaggcttatggtattacttaaacgaatcaggttcaatggctactggttgggttaaagacaaaggcttatggtattacttaaatgaatcaggttcaatggctactggttggtttacagtttctggtaaatggtactatacctataattcaggagatttattagtaaacacgactacacccgatggctatcgagtcaatgctaacggtgagtgggtagga

SEQ ID NO: 5 - protein, 820 aa, Streptococcus pneumoniae
MGSYELGRHQAGQVKKESNRVSYIDGDQAGQKAENLTPDEVSKREGINAEQIVIKITDQGYVTSHGDHYHYYNGKVPYDAIISEELLMKDPNYQLKDSDIVNEIKGGYVIKVDGKYYVYLKDAAHADNIRTKEEIKRQKQEHSHNHNSRADNAVAAARAQGRYTTDDGYIFNASDIIEDTGDAYIVPHGDHYHYIPKNELSASELAAAEAYWNGKQGSRPSSSSSYNANPVQPRLSENHNLTVTPTYHQNQGENISSLLRELYAKPLSERHVESDGLIFDPAQITSRTARGVAVPHGNHYHFIPYEQMSELEKRIARIIPLRYRSNHWVPDSRPEQPSPQSTPEPSPSLQPAPNPQPAPSNPIDEKLVKEAVRKVGDGYVFEENGVSRYIPAKDLSAETAAGIDSKLAKQESLSHKLGAKKTDLPSSDREFYNKAYDLLARIHQDLLDNKGRQVDFEVLDNLLERLKDVSSDKVKLVDDILAFLAPIRHPERLGKPNAQITYTDDEIQVAKLAGKYTTEDGYIFDPRDITSDEGDAYVTPHMTHSHWIKKDSLSEAERAAAQAYAKEKGLTPPSTDHQDSGNTEAKGAEAIYNRVKAAKKVPLDRMPYNLQYTVEVKNGSLIIPHYDHYHNIKFEWFDEGLYEAPKGYSLEDLLATVKYYVEHPNERPHSDNGFGNASDHVRKNKADQDSKPDEDKEHDEVSEPTHPESDEKENHAGLNPSADNLYKPSTDTEETEEEAEDTTDEAEIPQVENSVINAKIADAEALLEKVTDPSIRQNAMETLTGLKSSLLLGTKDNNTISAEVDSLLALLKESQPAPIQ

SEQ ID NO: 6 - DNA, 2463 bp, Streptococcus pneumoniae
atgggatcctatgaacttggtcgtcaccaagctggtcaggttaagaaagagtctaatcgagtttcttatatagatggtgatcaggctggtcaaaaggcagaaaatttgacaccagatgaagtcagtaagagagaggggatcaacgccgaacaaattgttatcaagattacggatcaaggttatgtgacctctcatggagaccattatcattactataatggcaaggttccttatgatgccatcatcagtgaagaacttctcatgaaagatccgaattatcagttgaaggattcagacattgtcaatgaaatcaagggtggctatgtgattaaggtagacggaaaatactatgtttaccttaaagatgcggcccatgcggacaatattcggacaaaagaagagattaaacgtcagaagcaggaacacagtcataatcataactcaagagcagataatgctgttgctgcagccagagcccaaggacgttatacaacggatgatgggtatatcttcaatgcatctgatatcattgaggacacgggtgatgcttatatcgttcctcacggcgaccattaccattacattcctaagaatgagttatcagctagcgagttagctgctgcagaagcctattggaatgggaagcagggatctcgtccttcttcaagttctagttataatgcaaatccagttcaaccaagattgtcagagaaccacaatctgactgtcactccaacttatcatcaaaatcaaggggaaaacatttcaagccttttacgtgaattgtatgctaaacccttatcagaacgccatgtagaatctgatggccttattttcgacccagcgcaaatcacaagtcgaaccgccagaggtgtagctgtccctcatggtaaccattaccactttatcccttatgaacaaatgtctgaattggaaaaacgaattgctcgtattattccccttcgttatcgttcaaaccattgggtaccagattcaagaccagaacaaccaagtccacaatcgactccggaacctagtccaagtctgcaacctgcaccaaatcctcaaccagctccaagcaatccaattgatgagaaattggtcaaagaagctgttcgaaaagtaggcgatggttatgtctttgaggagaatggagtttctcgttatatcccagccaaggatctttcagcagaaacagcagcaggcattgatagcaaactggccaagcaggaaagtttatctcataagctaggagctaagaaaactgacctcccatctagtgatcgagaattttacaataaggcttatgacttactagcaagaattcaccaagatttacttgataataaaggtcgacaagttgattttgaggttttggataacctgttggaacgactcaaggatgtctcaagtgataaagtcaagttagtggatgatattcttgccttcttagctccgattcgtcatccagaacgtttaggaaaaccaaatgcgcaaattacctacactgatgatgagattcaagtagccaagttggcaggcaagtacacaacagaagacggttatatctttgatcctcgtgatataaccagtgatgagggggatgcctatgtaactccacatatgacccatagccactggattaaaaaagatagtttgtctgaagctgagagagcggcagcccaggcttatgctaaagagaaaggtttgacccctccttcgacagaccatcaggattcaggaaatactgaggcaaaaggagcagaagctatctacaaccgcgtgaaagcagctaagaaggtgccacttgatcgtatgccttacaatcttcaatatactgtagaagtcaaaaacggtagtttaatcatacctcattatgaccattaccataacatcaaatttgagtggtttgacgaaggcctttatgaggcacctaaggggtatagtcttgaggatcttttggcgactgtcaagtactatgtcgaacatccaaacgaacgtccgcattcagataatggttttggtaacgctagtgaccatgttcgtaaaaataaggcagaccaagatagtaaacctgatgaagataaggaacatgatgaagtaagtgagccaactcaccctgaatctgatgaaaaagagaatcacgctggtttaaatccttcagcagataatctttataaaccaagcactgatacggaagagacagaggaagaagctgaagataccacagatgaggctgaaattcctcaagtagagaattctgttattaacgctaagatagcagatgcggaggccttgctagaaaaagtaacagatcctagtattagacaaaatgctatggagacattgactggtctaaaaagtagtcttcttctcggaacgaaagataataacactatttcagcagaagtagatagtctcttggctttgttaaaagaaagtcaaccggctcctatacagtag

SEQ ID NO: 7 - protein, 445 aa, Streptococcus pneumoniae
MADTPSSEVIKETKVGSIIQQNNIKYKVLTVEGNIGTVQVGNGVTPVEFEAGQDGKPFTIPTKITVGDKVFTVTEVASQAFSYYPDETGRIVYYPSSITIPSSIKKIQKKGFHGSKAKTIIFDKGSQLEKIEDRAFDFSELEEIELPASLEYIGTSAFSFSQKLKKLTFSSSSKLELISHEAFANLSNLEKLTLPKSVKTLGSNLFRLTTSLKHVDVEEGNESFASVDGVLFSKDKTQLIYYPSQKNDESYKTPKETKELASYSFNKNSYLKKLELNEGLEKIGTFAFADAIKLEEISLPNSLETIERLAFYGNLELKELILPDNVKNFGKHVMNGLPKLKSLTIGNNINSLPSFFLSGVLDSLKEIHIKNKSTEFSVKKDTFAIPETVKFYVTSEHIKDVLKSNLSTSNDIIVEKVDNIKQETDVAKPKKNSNQGVVGWVKDKG

SEQ ID NO: 8 - DNA, 1338 bp, Streptococcus pneumoniae
atggcagatactcctagttcggaagtaatcaaagagactaaagttggaagtattattcaacaaaataatatcaaatataaggttctaactgtagaaggtaacataggaactgttcaagtgggtaatggagttactcctgtagagtttgaagctggtcaagatggaaaaccattcacgattcctacaaaaatcacagtaggtgataaagtatttaccgttactgaagtagctagtcaagcttttagttattatccagatgaaacaggtagaattgtctactatcctagctctattactatcccatcaagcataaaaaaaatacaaaaaaaaggcttccatggaagtaaagctaaaactattatttttgacaaaggcagtcagctggagaaaattgaagatagagcttttgatttttctgaattagaagagattgaattgcctgcatctctagaatatattggaacaagtgcattttcttttagtcaaaaattgaaaaagctaaccttttcctcaagttcaaaattagaattaatatcacatgaggcttttgctaatttatcaaatttagagaaactaacattaccaaaatcggttaaaacattaggaagtaatctatttagactcactactagcttaaaacatgttgatgttgaagaaggaaatgaatcgtttgcctcagttgatggtgttttgttttcaaaagataaaactcaattaatttattatccaagtcaaaaaaatgacgaaagttataaaacgcctaaggagacaaaagaacttgcatcatattcgtttaataaaaattcttacttgaaaaaactcgaattgaatgaaggtttagaaaaaatcggtacttttgcatttgcggatgcgattaaacttgaagaaattagcttaccaaatagtttagaaactattgaacgtttagccttttacggtaatttagaattaaaagaacttatattaccagataatgttaaaaattttggtaaacacgttatgaacggtttaccaaaattaaaaagtttaacaattggtaataatatcaactcattgccgtccttcttcctaagtggcgtcttagattcattaaaggaaattcatattaagaataaaagtacagagttttctgtgaaaaaagatacatttgcaattcctgaaactgttaagttctatgtaacatcagaacatataaaagatgttcttaaatcaaatttatctactagtaatgatatcattgttgaaaaagtagataatataaaacaagaaactgatgtagctaaacctaaaaagaattctaatcagggagtagttggttgggttaaagacaaaggttaa

SEQ ID NO: 9 - protein, 471 aa, Streptococcus pneumoniae
MANKAVNDFILAMNYDKKKLLTHQGESIENRFIKEGNQLPDEFVVIERKKRSLSTNTSDISVTACNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILCGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIREATGLAWEWWRTVYEKTDLPLVRKRTISIWGTTLYPQVEDKVEND

SEQ ID NO: 10 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 11 - DNA, 37 bp, Streptococcus pneumoniae
ctagccatgggatcctatgaacttggtcgtcaccaag

SEQ ID NO: 12 - DNA, 31 bp, Streptococcus pneumoniae
agtcctcgagctactgtataggagccggttg

SEQ ID NO: 13 - DNA, 45 bp, Streptococcus pneumoniae
tagcctcgagttaacctttaacccaaccaactactccctgattag

SEQ ID NO: 14 - DNA, 44 bp, Streptococcus pneumoniae
ctaatgaaccacatatggcagatactcctagttcggaagtaatc